SEQUENCE LISTING (97 sequences)

SEQ ID NO: 1 - protein, 7 aa, Artificial Sequence
  Description of Artificial Sequence Synthetic Peptide
KRRQRRR

SEQ ID NO: 2 - protein, 16 aa, Artificial Sequence
  Description of Artificial Sequence Synthetic Peptide
RQIKIWFQNRRMKWKK

SEQ ID NO: 3 - protein, 34 aa, Artificial Sequence
  Description of Artificial Sequence Synthetic Peptide
DAATATRGRSAASRPTERPRAPARSASRPRRPVD

SEQ ID NO: 4 - protein, 16 aa, Artificial Sequence
  Description of Artificial Sequence Synthetic Peptide
AAVALLPAVLLALLAP

SEQ ID NO: 5 - protein, 16 aa, Artificial Sequence
  Description of Artificial Sequence Synthetic Peptide
AAVLLPVLLPVLLAAP

SEQ ID NO: 6 - protein, 15 aa, Artificial Sequence
  Description of Artificial Sequence Synthetic Peptide
VTVLALGALAGVGVG

SEQ ID NO: 7 - protein, 17 aa, Artificial Sequence
  Description of Artificial Sequence Synthetic Peptide
GALFLGWLGAAGSTMGA

SEQ ID NO: 8 - protein, 17 aa, Artificial Sequence
  Description of Artificial Sequence Synthetic Peptide
MGLGLHLLVLAAALQGA

SEQ ID NO: 9 - protein, 24 aa, Artificial Sequence
  Description of Artificial Sequence Synthetic Peptide
LGTYTQDFNKFHTFPQTAIGVGAP

SEQ ID NO: 10 - protein, 26 aa, Artificial Sequence
  Description of Artificial Sequence Synthetic Peptide
GWTLNSAGYLLKINLKALAALAKKIL

SEQ ID NO: 11 - protein, 16 aa, Artificial Sequence
  Description of Artificial Sequence Synthetic Peptide
TPPKKKRKVEDPKKKK

SEQ ID NO: 12 - protein, 7 aa, Artificial Sequence
  Description of Artificial Sequence Synthetic Peptide
RRRRRRR

SEQ ID NO: 13 - protein, 18 aa, Artificial Sequence
  Description of Artificial Sequence Synthetic Peptide
KLALKLALKALKAALKLA

SEQ ID NO: 14 - protein, 16 aa, Artificial Sequence
  Description of Artificial Sequence Synthetic Peptide
GLFGAIAGFIENGWEG

SEQ ID NO: 15 - protein, 16 aa, Artificial Sequence
  Description of Artificial Sequence Synthetic Peptide
FFGAVIGTIALGVATA

SEQ ID NO: 16 - protein, 16 aa, Artificial Sequence
  Description of Artificial Sequence Synthetic Peptide
FLGFLLGVGSAIASGV

SEQ ID NO: 17 - protein, 16 aa, Artificial Sequence
  Description of Artificial Sequence Synthetic Peptide
GVFVLGFLGFLATAGS

SEQ ID NO: 18 - protein, 16 aa, Artificial Sequence
  Description of Artificial Sequence Synthetic Peptide
GAAIGLAWIPYFGPAA

SEQ ID NO: 19 - protein, 56 aa, Artificial Sequence
  Description of Artificial Sequence Synthetic Peptide
ACTCPYCKDSEGRGSGDPGKKKQHICHIQGCGKVYGKTSHLRAHLRWHTGERPFMC

SEQ ID NO: 20 - protein, 54 aa, Artificial Sequence
  Description of Artificial Sequence Synthetic Peptide
ACTCPNCKDGEKRSGEQGKKKHVCHIPDCGKTFRKTSLLRAHVRLHTGERPFVC

SEQ ID NO: 21 - protein, 55 aa, Artificial Sequence
  Description of Artificial Sequence Synthetic Peptide
ACTCPNCKEGGGRGTNLGKKKQHICHIPGCGKVYGKTSHLRAHLRWHSGERPFVC

SEQ ID NO: 22 - protein, 56 aa, Artificial Sequence
  Description of Artificial Sequence Synthetic Peptide
ACSCPNCREGEGRGSNEPGKKKQHICHIEGCGKVYGKTSHLRAHLRWHTGERPFIC

SEQ ID NO: 23 - protein, 60 aa, Artificial Sequence
  Description of Artificial Sequence Synthetic Peptide
RCTCPNCTNEMSGLPPIVGPDERGRKQHICHIPGCERLYGKASHLKTHLRWHTGERPFLC

SEQ ID NO: 24 - protein, 58 aa, Artificial Sequence
  Description of Artificial Sequence Synthetic Peptide
TCDCPNCQEAERLGPAGVHLRKKNIHSCHIPGCGKVYGKTSHLKAHLRWHTGERPFVC

SEQ ID NO: 25 - protein, 53 aa, Artificial Sequence
  Description of Artificial Sequence Synthetic Peptide
RCTCPNCKAIKHGDRGSQHTHLCSVPGCGKTYKKTSHLRAHLRKHTGDRPFVC

SEQ ID NO: 26 - protein, 56 aa, Artificial Sequence
  Description of Artificial Sequence Synthetic Peptide
PQISLKKKIFFFIFSNFRGDGKSRIHICHLCNKTYGKTSHLRAHLRGHAGNKPFAC

SEQ ID NO: 27 - protein, 31 aa, Artificial Sequence
  Description of Artificial Sequence Synthetic Peptide
WWETWKPFQCRICMRNFSTRQARRNHRRRHR

SEQ ID NO: 28 - protein, 16 aa, Artificial Sequence
  Description of Artificial Sequence Synthetic Peptide
GKINLKALAALAKKIL

SEQ ID NO: 29 - protein, 16 aa, Artificial Sequence
  Description of Artificial Sequence Synthetic Peptide
RVIRVWFQNKRCKDKK

SEQ ID NO: 30 - protein, 39 aa, Artificial Sequence
  Description of Artificial Sequence Synthetic Peptide
GRKKRRQRRRPPQGRKKRRQRRRPPQGRKKRRQRRRPPQ

SEQ ID NO: 31 - protein, 22 aa, Artificial Sequence
  Description of Artificial Sequence Synthetic Peptide
GEQIAQLIAGYIDIILKKKKSK

SEQ ID NO: 32 - protein, 14 aa, Artificial Sequence
  Description of Artificial Sequence Synthetic Peptide
GRKKRRQRRRPPQC

SEQ ID NO: 33 - protein, 28 aa, Artificial Sequence
  Description of Artificial Sequence Synthetic Peptide
  N-Term maleimide
  C-Term amide
AAVALLPAVLLALLAPRKKRRQRRRPPQ

SEQ ID NO: 34 - protein, 29 aa, Artificial Sequence
  Description of Artificial Sequence Synthetic Peptide
AAVALLPAVLLALLAPRKKRRQRRRPPQC

SEQ ID NO: 35 - protein, 28 aa, Artificial Sequence
  Description of Artificial Sequence Synthetic Peptide
  N-Term maleimide
  C-Term amide
AAVALLPAVLLALLAPRKKRRQRRRPPQ

SEQ ID NO: 36 - protein, 29 aa, Artificial Sequence
  Description of Artificial Sequence Synthetic Peptide
  C-Term amide
RKKRRQRRRPPQCAAVALLPAVLLALLAP

SEQ ID NO: 37 - protein, 12 aa, Artificial Sequence
  Description of Artificial Sequence Synthetic Peptide
  N-Term BrAc
  C-Term amide
GRKKRRQRRRPQ

SEQ ID NO: 38 - protein, 29 aa, Artificial Sequence
  Description of Artificial Sequence Synthetic Peptide
  N-Term BrAc
  C-Term amide
RRRQRRKRGGDIMGEWGNEIFGAIAGFLG

SEQ ID NO: 39 - protein, 29 aa, Artificial Sequence
  Description of Artificial Sequence Synthetic Peptide
  C-Term amide
RRRQRRKRGGDIMGEWGNEIFGAIAGFLG

SEQ ID NO: 40 - protein, 25 aa, Artificial Sequence
  Description of Artificial Sequence Synthetic Peptide
CYGRKKRRQRRRGYGRKKRRQRRRG

SEQ ID NO: 41 - protein, 13 aa, Artificial Sequence
  Description of Artificial Sequence Synthetic Peptide
  N-Term maleimide
  C-Term amide
GRKKRRQRRRPPQ

SEQ ID NO: 42 - protein, 16 aa, Artificial Sequence
  Description of Artificial Sequence Synthetic Peptide
  C-Term amide
KLWKAWPKLWKKLWKP

SEQ ID NO: 43 - protein, 22 aa, Artificial Sequence
  Description of Artificial Sequence Synthetic Peptide
  C-Term amide
AAVALLPAVLLALLAPRRRRRR

SEQ ID NO: 44 - protein, 16 aa, Artificial Sequence
  Description of Artificial Sequence Synthetic Peptide
  C-Term amide
RLWRALPRVLRRLLRP

SEQ ID NO: 45 - protein, 28 aa, Artificial Sequence
  Description of Artificial Sequence Synthetic Peptide
  C-Term amide
AAVALLPAVLLALLAPSGASGLDKRDYV

SEQ ID NO: 46 - protein, 28 aa, Artificial Sequence
  Description of Artificial Sequence Synthetic Peptide
  N-Term maleimide
  C-Term amide
AAVALLPAVLLALLAPSGASGLDKRDYV

SEQ ID NO: 47 - protein, 29 aa, Artificial Sequence
  Description of Artificial Sequence Synthetic Peptide
  C-Term amide
SGASGLDKRDYVAAVAALLPAVLLALLAP

SEQ ID NO: 48 - protein, 33 aa, Artificial Sequence
  Description of Artificial Sequence Synthetic Peptide
  C-Term amide
LLETLLKPFQCRICMRNFSTRQARRNHRRRHRR

SEQ ID NO: 49 - protein, 27 aa, Artificial Sequence
  Description of Artificial Sequence Synthetic Peptide
  C-Term amide
AAVACRICMRNFSTRQARRNHRRRHRR

SEQ ID NO: 50 - protein, 16 aa, Artificial Sequence
  Description of Artificial Sequence Synthetic Peptide
  N-Term maleimide
  C-Term amide
RQIKIWFQNRRMKWKK

SEQ ID NO: 51 - protein, 16 aa, Artificial Sequence
  Description of Artificial Sequence Synthetic Peptide
  C-Term amide
RQIKIWFQNRRMKWKK

SEQ ID NO: 52 - protein, 35 aa, Artificial Sequence
  Description of Artificial Sequence Synthetic Peptide
RQIKIWFQNRRMKWKKDIMGEWGNEIFGAIAGFLG

SEQ ID NO: 53 - protein, 37 aa, Artificial Sequence
  Description of Artificial Sequence Synthetic Peptide
  N-Term maleimide
  C-Term amide
SGRGKQGGKARAKAKTRSSRAGLQFPVGRVHRLLRKG

SEQ ID NO: 54 - protein, 38 aa, Artificial Sequence
  Description of Artificial Sequence Synthetic Peptide
  C-Term amide
SGRGKQGGKARAKAKTRSSRAGLQFPVGRVHRLLRKGC

SEQ ID NO: 55 - protein, 23 aa, Artificial Sequence
  Description of Artificial Sequence Synthetic Peptide
  C-Term amide
KGSKKAVTKAQKKDGKKRKRSRK

SEQ ID NO: 56 - protein, 25 aa, Artificial Sequence
  Description of Artificial Sequence Synthetic Peptide
  C-Term amide
KKDGKKRKRSRKESYSVYVYKVLKQ

SEQ ID NO: 57 - protein, 36 aa, Artificial Sequence
  Description of Artificial Sequence Synthetic Peptide
KGSKKAVTKAQKKDGKKRKRSRKESYSVYVYKVLKQ

SEQ ID NO: 58 - protein, 27 aa, Artificial Sequence
  Description of Artificial Sequence Synthetic Peptide
  N-Term BrAc
  C-Term amide
GWTLNSAGYLLGKINLKALAALAKKIL

SEQ ID NO: 59 - protein, 18 aa, Artificial Sequence
  Description of Artificial Sequence Synthetic Peptide
  C-Term amide
KLALKLALKALKAALKLA

SEQ ID NO: 60 - protein, 18 aa, Artificial Sequence
  Description of Artificial Sequence Synthetic Peptide
  N-Term BrAc
  C-Term amide
KLALKLALKALKAALKLA

SEQ ID NO: 61 - protein, 21 aa, Artificial Sequence
  Description of Artificial Sequence Synthetic Peptide
  N-Term Ac
  C-Term amide
KETWWETWWTEWSQPKKKRKV

SEQ ID NO: 62 - protein, 28 aa, Artificial Sequence
  Description of Artificial Sequence Synthetic Peptide
  C-Term amide
KETWWETWWTEWSQPGRKKRRQRRRPPQ

SEQ ID NO: 63 - protein, 7 aa, Artificial Sequence
  Description of Artificial Sequence Synthetic Peptide
  N-Term BrAc
RRRRRRR

SEQ ID NO: 64 - protein, 10 aa, Artificial Sequence
  Description of Artificial Sequence Synthetic Peptide
QQQQQQQQQQ

SEQ ID NO: 65 - protein, 20 aa, Artificial Sequence
  Description of Artificial Sequence Synthetic Peptide
  C-Term amide
RRRQRRKRGGQQQQQQQQQQ

SEQ ID NO: 66 - protein, 15 aa, Artificial Sequence
  Description of Artificial Sequence Synthetic Peptide
  C-Term amide
RVIRWFQNKRCKDKK

SEQ ID NO: 67 - protein, 18 aa, Artificial Sequence
  Description of Artificial Sequence Synthetic Peptide
  N-Term Ac
  C-Term amide
LGLLLRHLRHHSNLLANI

SEQ ID NO: 68 - protein, 18 aa, Artificial Sequence
  Description of Artificial Sequence Synthetic Peptide
  C-Term amide
GQMSEIEAKVRTVKLARS

SEQ ID NO: 69 - protein, 16 aa, Artificial Sequence
  Description of Artificial Sequence Synthetic Peptide
  C-Term amide
KLWSAWPSLWSSLWKP

SEQ ID NO: 70 - protein, 9 aa, Artificial Sequence
  Description of Artificial Sequence Synthetic Peptide
  C-Term amide
KKKKKKKKK

SEQ ID NO: 71 - protein, 21 aa, Artificial Sequence
  Description of Artificial Sequence Synthetic Peptide
  C-Term amide
AARLHRFKNKGKDSTEMRRRR

SEQ ID NO: 72 - protein, 22 aa, Artificial Sequence
  Description of Artificial Sequence Synthetic Peptide
  N-Term maleimide
  C-Term amide
GLGSLLKKAGKKLKQPKSKRKV

SEQ ID NO: 73 - protein, 4 aa, Artificial Sequence
  Description of Artificial Sequence Synthetic Peptide
  MOD_RES (1): DiMeY
  N-Term maleimide
  C-Term amide
XRFK

SEQ ID NO: 74 - protein, 14 aa, Artificial Sequence
  Description of Artificial Sequence Synthetic Peptide
  MOD_RES (1): DiMeY
  N-Term maleimide
  C-Term amide
XRFKQQQQQQQQQQ

SEQ ID NO: 75 - protein, 4 aa, Artificial Sequence
  Description of Artificial Sequence Synthetic Peptide
  N-Term maleimide
  C-Term amide
WRFK

SEQ ID NO: 76 - protein, 14 aa, Artificial Sequence
  Description of Artificial Sequence Synthetic Peptide
  N-Term maleimide
  C-Term amide
WRFKQQQQQQQQQQ

SEQ ID NO: 77 - protein, 4 aa, Artificial Sequence
  Description of Artificial Sequence Synthetic Peptide
  N-Term maleimide
  C-Term amide
YRFK

SEQ ID NO: 78 - protein, 12 aa, Artificial Sequence
  Description of Artificial Sequence Synthetic Peptide
  N-Term maleimide
  C-Term amide
YRFKYRFKYRFK

SEQ ID NO: 79 - protein, 10 aa, Artificial Sequence
  Description of Artificial Sequence Synthetic Peptide
  N-Term maleimide
  C-Term amide
WRFKKSKRKV

SEQ ID NO: 80 - protein, 20 aa, Artificial Sequence
  Description of Artificial Sequence Synthetic Peptide
  N-Term maleimide
  C-Term amide
WRFKAAVALLPAVLLALLAP

SEQ ID NO: 81 - protein, 4 aa, Artificial Sequence
  Description of Artificial Sequence Synthetic Peptide
  MOD_RES (1): DiMeY
  C-Term amide
XRFK

SEQ ID NO: 82 - protein, 4 aa, Artificial Sequence
  Description of Artificial Sequence Synthetic Peptide
  C-Term amide
YRFK

SEQ ID NO: 83 - protein, 4 aa, Artificial Sequence
  Description of Artificial Sequence Synthetic Peptide
  MOD_RES (1): DiMeY
  C-Term amide
XRFK

SEQ ID NO: 84 - protein, 4 aa, Artificial Sequence
  Description of Artificial Sequence Synthetic Peptide
  C-Term amide
WRFK

SEQ ID NO: 85 - protein, 4 aa, Artificial Sequence
  Description of Artificial Sequence Synthetic Peptide
  MOD_RES (1): DiMeY
  C-Term amide
XRWK

SEQ ID NO: 86 - protein, 4 aa, Artificial Sequence
  Description of Artificial Sequence Synthetic Peptide
  MOD_RES (4): DiMeY
  C-Term amide
KFRX

SEQ ID NO: 87 - protein, 8 aa, Artificial Sequence
  Description of Artificial Sequence Synthetic Peptide
  N-Term maleimide
  C-Term amide
WRFKWRFK

SEQ ID NO: 88 - protein, 12 aa, Artificial Sequence
  Description of Artificial Sequence Synthetic Peptide
  N-Term maleimide
  C-Term amide
WRFKWRFKWRFK

SEQ ID NO: 89 - protein, 16 aa, Artificial Sequence
  Description of Artificial Sequence Synthetic Peptide
GKINLKALAALAKKIL

SEQ ID NO: 90 - protein, 16 aa, Artificial Sequence
  Description of Artificial Sequence Synthetic Peptide
RVIRVWFQNKRCKDKK

SEQ ID NO: 91 - protein, 39 aa, Artificial Sequence; identical to SEQ ID NO: 30
  Description of Artificial Sequence Synthetic Peptide

SEQ ID NO: 92 - protein, 22 aa, Artificial Sequence
  Description of Artificial Sequence Synthetic Peptide
GEQIAQLIAGYIDIILKKKKSK

SEQ ID NO: 93 - protein, 31 aa, Artificial Sequence; identical to SEQ ID NO: 27
  Description of Artificial Sequence Synthetic Peptide

SEQ ID NO: 94 - protein, 36 aa, Artificial Sequence; identical to SEQ ID NO: 57
  Description of Artificial Sequence Synthetic Peptide

SEQ ID NO: 95 - DNA, 20 bp, Artificial Sequence
  Description of Combined DNA/RNA Molecule Synthetic oligonucleotide
  Description of Artificial Sequence Synthetic oligonucleotide
gggucggaacccaaguuatt

SEQ ID NO: 96 - DNA, 21 bp, Artificial Sequence
  Description of Combined DNA/RNA Molecule Synthetic oligonucleotide
  Description of Artificial Sequence Synthetic oligonucleotide
uaagcuuggguuccgacccta

SEQ ID NO: 97 - protein, 23 aa, Artificial Sequence
  Description of Artificial Sequence Synthetic Peptide
  MOD_RES (2)..(5): this region may encompass 2 or 4 variable residues
  MOD_RES (7)..(18): variable residue
  MOD_RES (20)..(22): variable residue
CXXXXCXXXXXXXXXXXXHXXXH

(19) United States
(12) Patent Application Publication
(10) Pub. No.: US 2007/0213257 A1
(43) Pub. Date: Sep. 13, 2007

(54) COMPOSITIONS AND METHODS FOR COMPLEXES OF NUCLEIC ACIDS AND PEPTIDES

(75) Inventor: David S. Sweedler, Louisville, CO (US)

Correspondence Address: NASTECH PHARMACEUTICAL COMPANY INC, 3830 MONTE VILLA PARKWAY, BOTHELL, WA, 98021-7266, US

(73) Assignee: Nastech Pharmaceutical Company Inc.

(21) Appl. No.: 11/464,167

(22) Filed: Aug. 11, 2006

Related U.S. Application Data
(60) Provisional application No. 60/707,850, filed on Aug. 12, 2005.

(51) Int. Cl. A61K 48/00; A61K 38/16

(52) U.S. Cl. 514/7; 514/44

(57) ABSTRACT

Compositions and methods are provided for producing a complex between a double stranded (ds) nucleic acid and a peptide, comprising: a. solubilizing the nucleic acid in an aqueous solution; b. solubilizing the peptide in an aqueous solution; and c. mixing the solubilized ds nucleic acid and the solubilized peptide in the presence of an organic salt.

This application claims the benefit under 35 U.S.C. § 119(e) of U.S. Provisional Application No. 60/707,850 filed Aug. 12, 2005, which is incorporated herein by reference.

### BACKGROUND OF THE INVENTION

Delivering nucleic acids into animal and plant cells has long been an important object of molecular biology research and development. Recent developments in the areas of gene therapy, antisense therapy and RNA interference (RNAi) therapy have created a need to develop more efficient means for introducing nucleic acids into cells.

RNA interference is a process of sequence-specific post transcriptional gene silencing in cells initiated by a double-stranded (ds) polynucleotide, usually a dsRNA, that is homologous in sequence to a portion of a targeted messenger RNA (mRNA). Introduction of a suitable dsRNA into cells leads to destruction of endogenous, cognate mRNAs (i.e., mRNAs that share substantial sequence identity with the introduced dsRNA). The dsRNA molecules are cleaved by an RNase III family nuclease called dicer into short-interfering RNAs (siRNAs), which are 19-23 nucleotides (nt) in length. The siRNAs are then incorporated into a multicomponent nuclease complex known as the RNA-induced silencing complex or “RISC”. The RISC identifies mRNA substrates through their homology to the siRNA, and effectuates silencing of gene expression by binding to and destroying the targeted mRNA.

RNA interference is emerging a promising technology for modifying expression of specific genes in plant and animal cells, and is therefore expected to provide useful tools to treat a wide range of diseases and disorders amenable to treatment by modification of endogenous gene expression.

A variety of methods are available for delivering nucleic acid artificially into cells. These include transfection via calcium phosphate, cationic lipid, and lipsomal delivery. Nucleic acids can also be introduced into cells by electroporation and viral transduction. However, there are disadvantages to these methods. With viral gene delivery, there is a possibility that the replication deficient virus used as a delivery vehicle may revert to wild-type thus becoming pathogenic. Electroporation suffers from poor gene-transfer efficiency and therefore has limited clinical application. Finally, transfection may also be limited by poor efficiency and toxicity.

Synthetic and biological polypeptides show great potential as a tool to introduce nucleic acids into cells. However, synthetic peptides may elicit an undesired immune response and may be toxic because it is not be readily susceptible to degradation in the cell.

Biological peptides, i.e., fragments of naturally occurring proteins, typically do not suffer from the same disadvantages as synthetic peptides. Nonetheless, both biological and synthetic peptides can suffer from non-specific promiscuous aggregation when complexed with nucleic acids at physiological salt concentrations. Consequently, this instability severely limits the effectiveness of delivery of the nucleic acid via the polypeptide. Therefore, there remains a need for improved methods and formulations to deliver siNAs in an effective amount, in an active and enduring state, and using non-toxic delivery vehicles, to selected cells, tissues, or compartments to mediate regulation of gene expression in a manner that will alter a phenotype or disease state of the targeted cells.

### SUMMARY OF THE INVENTION

One aspect of the invention is a method for producing a complex between a double stranded (ds) nucleic acid and a peptide, comprising:

(a) solubilizing the nucleic acid in an aqueous solution;

(b) solubilizing the peptide in an aqueous solution; and

(c) mixing the solubilized ds nucleic acid and the solubilized peptide in the presence of an organic salt, in which steps a, b and c may be performed in any order, or simultaneously. One embodiment is the method, in which the ds nucleic acid is a dsRNA. In a related embodiment the dsRNA is siRNA. In a specific embodiment the siRNA has 29-50 base pairs, for example a dsRNA comprised of a sequence that is complementary to a region of a TNF-alpha gene. In an alternate embodiment, the ds nucleic acid is a dsDNA.

Another embodiment of the invention is the method for producing a complex between a double stranded (ds) nucleic acid and a peptide, in which the peptide is a polynucleotide delivery-enhancing polypeptide. In a related embodiment, the polynucleotide delivery-enhancing polypeptide comprises a histone protein, or a polypeptide or peptide fragment, derivative, analog, or conjugate thereof. In alternate embodiments, the polynucleotide delivery-enhancing polypeptide comprises an amphipathic amino acid sequence; a protein transduction domain or motif, or a fusogenic peptide domain or motif. In another alternate embodiment, polynucleotide delivery-enhancing polypeptide comprises a nucleic acid-binding domain or motif In this embodiment, the peptide may bind ds nucleic acid with a Kd less than about 100 nM, preferably less than about 10 nM. In another embodiment, the polynucleotide delivery-enhancing polypeptide selected from the group consisting of:

(SEQ ID NO:)

GRKKRRQRRRPPQC

(SEQ ID NO:)

Maleimide-AAVALLPAVLLALLAPRKKRRQRRRPPQ-amide

(SEQ ID NO:)

AAVALLPAVLLALLAPRKKRRQRRRPPQC

(SEQ ID NO:)

Maleimide-AAVALLPAVLLALLAPRKKRRQRRRPPQ-amide

(SEQ ID NO:)

NH2-RKKRRQRRRPPQCAAVALLPAVLLALLAP-amide

(SEQ ID NO:)

BrAc-GRKKRRQRRRPQ-amide

(SEQ ID NO:)

BrAc-RRRQRRKRGGDIMGEWGNEIFGAIAGFLGamide

(SEQ ID NO:)

NH2-RRRQRRKRGGDIMGEWGNEIFGAIAGFLG-amide

(SEQ ID NO:)

C(YGRKKRRQRRRG)2

(SEQ ID NO:)

Maleimide-GRKKRRQRRRPPQ-amide

(SEQ ID NO:)

NH2-KLWKAWPKLWKKLWKP-amide

(SEQ ID NO:)

AAVALLPAVLLALLAPRRRRRR-amide

(SEQ ID NO:)

RLWRALPRVLRRLLRP-amide

(SEQ ID NO:)

NH2-AAVALLPAVLLALLAPSGASGLDKRDYV-amide

(SEQ ID NO:)

Maleimide-AAVALLPAVLLALLAPSGASGLDKRDYV-amide

(SEQ ID NO:)

NH2-SGASGLDKRDYVAAVAALLPAVLLALLAP-amide

(SEQ ID NO:)

NH2-LLETLLKPFQCRICMRNFSTRQARRNHRRRHRR-amide

(SEQ ID NO:)

NH2-AAVACRICMRNFSTRQARRNHRRRHRR-amide

(SEQ ID NO:)

Maleimide-RQIKIWFQNRRMKWKK-amide

(SEQ ID NO:)

RQIKIWFQNRRMKWKK amide

(SEQ ID NO:)

NH2-RQIKIWFQNRRMKWKKDIMGEWGNEIFGAIAGFLG-amide

(SEQ ID NO:)

Maleimide-SGRGKQGGKARAKAKTRSSRAGLQFPVGRVHRLLRKG-
amide

(SEQ ID NO:)

SGRGKQGGKARAKAKTRSSRAGLQFPVGRVHRLLRKGC-amide

(SEQ ID NO:)

KGSKKAVTKAQKKDGKKRKRSRK-amide

(SEQ ID NO:)

NH2-KKDGKKRKRSRKESYSVYVYKVLKQ-amide

(SEQ ID NO:)

KGSKKAVTKAQKKDGKKRKRSRKESYSVYVYKVLKQ

(SEQ ID NO:)

BrAc-GWTLNSAGYLLGKINLKALAALAKKILamide

(SEQ ID NO:)

KLALKLALKALKAALKLAamide

(SEQ ID NO:)

BrAc-KLALKLALKALKAALKLAamide

(SEQ ID NO:)

Ac-KETWWETWWTEWSQPKKKRKV-amide

(SEQ ID NO:)

NH2-KETWWETWWTEWSQPGRKKRRQRRRPPQ-amide

(SEQ ID NO:)

BrAc-RRRRRRR

(SEQ ID NO:)

QqQqQqQqQq

(SEQ ID NO:)

NH2-RRRQRRKRGGqQqQqQqQqQ-amide

(SEQ ID NO:)

RVIRWFQNKRCKDKK-amide

(SEQ ID NO:)

Ac-LGLLLRHLRHHSNLLANI-amide

(SEQ ID NO:)

GQMSEIEAKVRTVKLARS-amide

(SEQ ID NO:)

NH2-KLWSAWPSLWSSLWKP-amide

(SEQ ID NO:)

NH2-KKKKKKKKK-amide

(SEQ ID NO:)

NH2-AARLHRFKNKGKDSTEMRRRR-amide

(SEQ ID NO:)

Maleimide-GLGSLLKKAGKKLKQPKSKRKV-amide

(SEQ ID NO:)

Maleimide-Dmt-r-FK-amide

(SEQ ID NO:)

Maleimide-Dmt-r-FKQqQqQqQqQq-amide

(SEQ ID NO:)

Maleimide-WRFK-amide

(SEQ ID NO:)

Maleimide-WRFKQqQqQqQqQq-amide

(SEQ ID NO:)

Maleimido-YRFK-amide

(SEQ ID NO:)

Maleimide-YRFKYRFKYRFK-amide

(SEQ ID NO:)

Maleimide-WRFKKSKRKV-amide

(SEQ ID NO:)

Maleimide-WRFKAAVALLPAVLLALLAP-amide

(SEQ ID NO:)

NH2-DiMeYrFKamide

(SEQ ID NO:)

NH2-YrFKamide

(SEQ ID NO:)

NH2-DiMeYRFKamide

(SEQ ID NO:)

NH2-WrFKamide

(SEQ ID NO:)

NH2-DiMeYrWKamide

(SEQ ID NO:)

NH2-KFrDiMeY-amide

(SEQ ID NO:)

Maleimide-WRFKWRFK-amide
and

(SEQ ID NO:)

Maleimide-WRFKWRFKWRFK-amide

Another embodiment of the invention is the method for producing a complex between a double stranded (ds) nucleic acid and a peptide, in which the polynucleotide delivery-enhancing polypeptide comprises one or more peptides selected from the group consisting of: histone H1, histone H2B, histone H3, and histone H4, or a fragment thereof, GKINLKALAALAKKIL, RVIRVWFQNKRCKDKK, GRKKRRQRRRPPQGRKKRRQRRRPPQGRKKRRQRRRPPQ, GEQIAQLIAGYIDIILKKKKSK, WWETWKPFQCRICMRNFSTRQARRNHRRRHR, Poly Lys-Trp (4:1, MW 20,000-50,000), Poly Orn-Trp (4:1, MW 20,000-50,000); and mellitin, preferably KGSKKAVTKAQKKDGKKRKRSRKESYSVYVYKVLKQ (PN73).

Another embodiment of the invention is the method for producing a complex between a double stranded (ds) nucleic acid and a peptide, in which the organic salt is selected from the group consisting of sugar amines and acids, peptide amines and acids, biocompatible salts, hydrophilic salts, and salts naturally occurring in man. In one embodiment, the organic salt is an organic cation, e.g., ammonium hydroxide, D-arginine, L-arginine, t-butylamine, calcium DL-malate, choline, ethanolamine, ethylenediamine, glycine, L-histidine, L-lysine, magnesium hydroxide, N-methyl-D-glucamine, L-ornithine hydrochloride, procaine hydrochloride, L-proline, pyridoxine, L-serine, sodium hydroxide, DL-triptophan, tromethamine, L-tyrosine, L-valine, carnitine, taurine, creatine malate, arginine alpha keto glutarate, ornithine alpha keto glutarate, spermine acetate, or spermidine chloride. In an alternate embodiment, the organic salt is an organic anion, e.g., acetic acid, adamantoic acid, alpha keto glutaric acid, D-aspartic acid, L-aspartic acid, benzenesulfonic acid, benzoic acid, 10-camphorsulfunic acid, citric acid, 1,2-ethanedisulfonic acid, fumaric acid, galacteric acid, D-gluconic acid, D-glucornic acid, glucaric acid, D-glutamic acid, L-glutamic acid, glutaric acid, glycolic acid, hippuric acid, hydrobromic acid, hydrochloric acid, 1-hydroxyl-2napthoic acid, lactobioinic acid, maleic acid, L-malic acid, mandelic acid, ethanesulfonic acid, mucic acid, 1,5 napthalenedisulfonic acid tetrahydrate, 2-napthalenesulfonic acid, nitric acid, oleic acid, pamoic acid, p-toluenesulfonic acid hydrate, D-saccharic acid monopotassium salt, salicylic acid, steric acid, succinic acid, sulfuric acid, tannic acid, D-tartaric acid, L-tartaric acid, or other related sugar carboxylate anions.

Other examples include aliphatic amines such as primary, secondary, tertiary and quaternary ammonium compounds from C4 through C22 aliphatics, preferably even numbered aliphatic hydrocarbons. Examples of primary aliphatic amines include N-propylamine; N-butylamine; N-pentylamine; N-hexylamine, N-heptylamine, N-octylamine, N-nonylamine; N-decylamine, N-dodecylamine; N-tetradecylamine; N-hexadecylamine, and N-octadecylamine.

Examples of secondary aliphatic amines include N,N-dipropylamine, N,N-dibutylamine; N,N-dipentylamine; N,N-dihexylamine, N,N-diheptylamine, N,N-dioctylamine, N,N-dinonylamine; N,N-didecylamine, N,N-didodecylamine; N,N,-ditetradecylamine; N,N-dihexadecylamine, and N,N-dioctadecylamine.

Examples of quaternary ammonium straight chain aliphatic amines include N-propyl-N,N,N-trimethylammonium chloride; N-butyl-N,N,N-trimethylammonium chloride, N-pentyl-N,N,N-trimethylammonium chloride; N-hexyl-N,N,N-trimethylammonium chloride; N-heptyl-N,N,N-trimethylammonium chloride; N-octyl-N,N,N-trimethylammonium chloride; N-nonyl-N,N,N-trimethylammonium chloride; N-decyl-N,N,N-trimethylammonium chloride; N-dodecyl-N,N,N-trimethylammonium chloride; N-tetradecyl-N,N,N-trimethylammonium chloride; N-hexadecyl-N,N,N-trimethylammonium chloride; and N-octadecyl-N,N,N-trimethylammonium chloride.

Examples of disubstituted symmetric quaternary aliphatic amines include N,N-dipropyl-N,N-dimethylammonium chloride; N,N-dibutyl-N,N-dimethylammonium chloride, N,N-dipentyl-N,N-dimethylammonium chloride; N,N-dihexyl-N,N-diimethylammonium chloride; N,N-diheptyl-N,N-diimethylammonium chloride; N,N-dioctyl-N,N-dimethylammonium chloride; N,N-dinonyl-N,N-dimethylammonium chloride; N,N-didecyl-N,N-dimethylammonium chloride; N,N-didodecyl-N,N-dimethylammonium chloride; N,N-ditetradecyl-N,N-dimethylammonium chloride; N,N-dihexadecyl-N,N-dimethylammonium chloride; and N,N-dioctadecyl-N,N-dimethylammonium chloride (DDAB).

Examples of unsaturated aliphatic quaternary ammonium cationic surfactant enes include N-1-propene-N,N,N-trimethylammonium chloride; N-1-butene-N,N,N-trimethylammonium chloride, N-1-pentene-N,N,N-trimethylammonium chloride; N-hexene-N,N,N-trimethylammonium chloride; N-heptene-N,N,N-trimethylammonium chloride; N-octene-N,N,N-trimethylammonium chloride; N-nonene-N,N,N-trimethylammonium chloride; N-decene-N,N,N-trimethylammonium chloride; N-dodecene-N,N,N-trimethylammonium chloride; N-tetradecene-N,N,N-trimethylammonium chloride; N-hexadecene-N,N,N-trimethylammonium chloride; and N-oleylamine-N,N,N-trimethylammonium chloride.

Examples of disubstituted symmetric quaternary unsaturated aliphatic amines include N,N-di-1-propene-N,N-dimethylammonium chloride; N,N-di-1-butene-N,N-dimethylammonium chloride, N,N-di-1-pentene-N,N-dimethylammonium chloride; N,N-dihexene-N,N-dimethylammonium chloride; N,N-diheptene-N,N-dimethylammonium chloride; N,N-dioctene-N,N-dimethylammonium chloride; N,N-dinonene-N,N-dimethylammonium chloride; N,N-decene-N,N-dimethylammonium chloride; N,N-didodecene-N,N-dimethylammonium chloride; N,N-tetradecene-N,N-dimethylammonium chloride; N,N-dihexadecene-N,N-dimethylammonium chloride; and N,N-dioleylamine-N,N-dimethylammonium chloride.

Other examples of organic cations include: DOTMA (N-[1-(2,3-dioleoyloxy) propyl]-N,N,N-trimethylammonium chloride, U.S. Pat. No. 4,897,355, DMRIE (D,L-1,2-0-dimyristyl-3-dimethylaminopropyl-b-hydroxyethylammoniumchloride, U.S. Pat. No. 5,264,618, DOTAP (1,2-bis (oleoyloxy)-3-3(trimethylammonia) propane) (Boehringer-Mannheim Catalog No. 1 202 375), DOGS (5-carboxysperminyglycine dioctadecylamide, U.S. Pat. No. 5,171,678 (TRANSFECTAM, Promega Corp., Madison, Wis.), DOSPA (2,3-dioleyloxy-N-[2(sperminecarboxyamido) ethyl}-N,N-dimethyl-1-propanaminium trifluoroacetate, U.S. Pat. No. 5,334,761, DDAB (Dimethyloctadecylammoniumbromide, U.S. Pat. No. 5,279,833, and TMTPS (N,N,N,N-Tetramethyltetrapalmylspermine, PCT Int. Pub. No. WO 95/17373. DOTMA, DOSPA, DDAB and TMTPS are sold by Life Technologies, Inc., Gaithersburg, Md., under the trade names LIPOFECTIN, LIPOFECTAMINE, LIPOFECTACE and CELLFECTIN,respectively. Other examples of organic cations are given in Ruysschaert et al., Biochem. Biophys. Res., 1994.

Another aspect of the invention is a composition consisting of a complex between a double stranded (ds) nucleic acid and a peptide, comprising: the ds nucleic acid, the peptide and an organic salt, wherein the peptide binds the ds nucleic acid with a Kd less than about 100 nM, and in which the complex is soluble in aqueous solution. In a related embodiment the dsRNA is siRNA. In a specific embodiment the siRNA has 29-50 base pairs, for example a dsRNA comprised of a sequence that is complementary to a region of a TNF-alpha gene. In an alternate embodiment, the ds nucleic acid is a dsDNA.

Another embodiment of the invention is the complex between a double stranded (ds) nucleic acid and a peptide, in which the peptide is a polynucleotide delivery-enhancing polypeptide. In a related embodiment, the polynucleotide delivery-enhancing polypeptide comprises a histone protein, or a polypeptide or peptide fragment, derivative, analog, or conjugate thereof. In alternate embodiments, the polynucleotide delivery-enhancing polypeptide comprises an amphipathic amino acid sequence; a protein transduction domain or motif; or a fusogenic peptide domain or motif. In another alternate embodiment, polynucleotide delivery-enhancing polypeptide comprises a nucleic acid-binding domain or motif. In this embodiment, the peptide may bind ds nucleic acid with a Kd less than about 100 nM, preferably less than about 10 nM. In another embodiment, the polynucleotide delivery-enhancing polypeptide selected from the group consisting of:

(SEQ ID NO:)

GRKKRRQRRRPPQC

(SEQ ID NO:)

Maleimide-AAVALLPAVLLALLAPRKKRRQRRRPPQ-amide

(SEQ ID NO:)

AAVALLPAVLLALLAPRKKRRQRRRPPQC

(SEQ ID NO:)

Maleimide-AAVALLPAVLLALLAPRKKRRQRRRPPQ-amide

(SEQ ID NO:)

NH2-RKKRRQRRRPPQCAAVALLPAVLLALLAP-amide

(SEQ ID NO:)

BrAc-GRKKRRQRRRPQ-amide

(SEQ ID NO:)

BrAc-RRRQRRKRGGDIMGEWGNEIFGAIAGFLGamide

(SEQ ID NO:)

NH2-RRRQRRKRGGDIMGEWGNEIFGAIAGFLG-amide

(SEQ ID NO:)

C(YGRKKRRQRRRG)2

(SEQ ID NO:)

Maleimide-GRKKRRQRRRPPQ-amide

(SEQ ID NO:)

NH2-KLWKAWPKLWKKLWKP-amide

(SEQ ID NO:)

AAVALLPAVLLALLAPRRRRRR-amide

(SEQ ID NO:)

RLWRALPRVLRRLLRP-amide

(SEQ ID NO:)

NH2-AAVALLPAVLLALLAPSGASGLDKRDYV-amide

(SEQ ID NO:)

Maleimide-AAVALLPAVLLALLAPSGASGLDKRDYV-amide

(SEQ ID NO:)

NH2-SGASGLDKRDYVAAVAALLPAVLLALLAP-amide

(SEQ ID NO:)

NH2-LLETLLKPFQCRICMRNFSTRQARRNHRRRHRR-amide

(SEQ ID NO:)

NH2-AAVACRICMRNFSTRQARRNHRRRHRR-amide

(SEQ ID NO:)

Maleimide-RQIKIWFQNRRMKWKK-amide

(SEQ ID NO:)

RQIKIWFQNRRMKWKK amide

(SEQ ID NO:)

NH2-RQIKIWFQNRRMKWKKDIMGEWGNEIFGAIAGFLG-amide

(SEQ ID NO:)

Maleimide-SGRGKQGGKARAKAKTRSSRAGLQFPVGRVHRLLRKG-
amide

(SEQ ID NO:)

SGRGKQGGKARAKAKTRSSRAGLQFPVGRVHRLLRKGC-amide

(SEQ ID NO:)

KGSKKAVTKAQKKDGKKRKRSRK-amide

(SEQ ID NO:)

NH2-KKDGKKRKRSRKESYSVYVYKVLKQ-amide

(SEQ ID NO:)

KGSKKAVTKAQKKDGKKRKRSRKESYSVYVYKVLKQ

(SEQ ID NO:)

BrAc-GWTLNSAGYLLGKINLKALAALAKKILamide

(SEQ ID NO:)

KLALKLALKALKAALKLAamide

(SEQ ID NO:)

BrAc-KLALKLALKALKAALKLAamide

(SEQ ID NO:)

Ac-KETWWETWWTEWSQPKKKRKV-amide

(SEQ ID NO:)

NH2-KETWWETWWTEWSQPGRKKRRQRRRPPQ-amide

(SEQ ID NO:)

BrAc-RRRRRRR

(SEQ ID NO:)

QqQqQqQqQq

(SEQ ID NO:)

NH2-RRRQRRKRGGqQqQqQqQqQ-amide

(SEQ ID NO:)

RVIRWFQNKRCKDKK-amide

(SEQ ID NO:)

Ac-LGLLLRHLRHHSNLLANI-amide

(SEQ ID NO:)

GQMSEIEAKVRTVKLARS-amide

(SEQ ID NO:)

NH2-KLWSAWPSLWSSLWKP-amide

(SEQ ID NO:)

NH2-KKKKKKKKK-amide

(SEQ ID NO:)

NH2-AARLHRFKNKGKDSTEMRRRR-amide

(SEQ ID NO:)

Maleimide-GLGSLLKKAGKKLKQPKSKRKV-amide

(SEQ ID NO:)

Maleimide-Dmt-r-FK-amide

(SEQ ID NO:)

Maleimide-Dmt-r-FKQqQqQqQqQq-amide

(SEQ ID NO:)

Maleimide-WRFK-amide

(SEQ ID NO:)

Maleimide-WRFKQqQqQqQqQq-amide

(SEQ ID NO:)

Maleimido-YRFK-amide

(SEQ ID NO:)

Maleimide-YRFKYRFKYRFK-amide

(SEQ ID NO:)

Maleimide-WRFKKSKRKV-amide

(SEQ ID NO:)

Maleimide-WRFKAAVALLPAVLLALLAP-amide

(SEQ ID NO:)

NH2-DiMeYrFKamide

(SEQ ID NO:)

NH2-YrFKamide

(SEQ ID NO:)

NH2-DiMeYRFKamide

(SEQ ID NO:)

NH2-WrFKamide

(SEQ ID NO:)

NH2-DiMeYrWKamide

(SEQ ID NO:)

NH2-KFrDiMeY-amide

(SEQ ID NO:)

Maleimide-WRFKWRFK-amide
and

(SEQ ID NO:)

Maleimide-WRFKWRFKWRFK-amide

Another embodiment of the invention is the complex between a double stranded (ds) nucleic acid and a peptide, in which the polynucleotide delivery-enhancing polypeptide comprises one or more peptides selected from the group consisting of: histone H1, histone H2B, histone H3, and histone H4, or a fragment thereof; GKINLKALAALAKKIL, RVIRVWFQNKRCKDKK, GRKKRRQRRRPPQGRKKRRQRRRPPQGRKKRRQRRRPPQ, GEQIAQLIAGYIDIILKKKKSK, WWETWKPFQCRICMRNFSTRQARRNHRRRHR, Poly Lys-Trp (4:1, MW 20,000-50,000), Poly Orn-Trp (4:1, MW 20,000-50,000); and mellitin, preferably KGSKKAVTKAQKKDGKKRKRSRKESYSVYVYKVLKQ (PN73).

Another embodiment of the invention is the complex between a double stranded (ds) nucleic acid and a peptide, in which the organic salt is selected from the group consisting of sugar amines and acids, peptide amines and acids, biocompatible salts, hydrophilic salts, and salts naturally occurring in man. In one embodiment, the organic salt is an organic cation, e.g., ammonium hydroxide, D-arginine, L-arginine, t-butylamine, calcium DL-malate, choline, diethanolamine, ethylenediamine, glycine, L-histidine, L-lysine, magnesium hydroxide, N-methyl-D-glucamine, L-ornithine hydrochloride, procaine hydrochloride, L-proline, pyridoxine, L-serine, sodium hydroxide, DL-triptophan, L-triptophan, tromethamine, L-tyrosine, L-valine, carnitine, taurine, creatine malate, arginine alpha keto glutarate, ornithine alpha keto glutarate, spermine acetate, or spermidine chloride. In an alternate embodiment, the organic salt is an organic anion, e.g., acetic acid, adamantoic acid, alpha keto glutaric acid, D-aspartic acid, L-aspartic acid, benzenesulfonic acid, benzoic acid, 10-camphorsulfunic acid, citric acid, 1,2-ethanedisulfonic acid, fumaric acid, galacteric acid, D-gluconic acid, D-glucornic acid, glucaric acid, D-glutamic acid, L-glutamic acid, glutaric acid, glycolic acid, hippuric acid, hydrobromic acid, hydrochloric acid, 1-hydroxyl-2napthoic acid, lactobioinic acid, maleic acid, L-malic acid, mandelic acid, ethanesulfonic acid, mucic acid, 1,5napthalenedisulfonic acid tetrahydrate, 2-napthalenesulfonic acid, nitric acid, oleic acid, pamoic acid, p-toluenesulfonic acid hydrate, D-saccharic acid monopotassium salt, salicylic acid, steric acid, succinic acid, sulfuric acid, tannic acid, D-tartaric acid, L-tartaric acid, or other related sugar carboxylate anions.

### DETAILED DESCRIPTION OF INVENTION

The present invention satisfies these needs and fulfills additional objects and advantages by providing novel compositions and methods that employ a short interfering nucleic acid (siNA), or a precursor thereof, in combination with a polynucleotide delivery-enhancing polypeptide and an organic counter-ion. The polynucleotide delivery-enhancing polypeptide is a natural or artificial polypeptide selected for its ability to enhance intracellular delivery or uptake of polynucleotides, including siNAs and their precursors. The counter-ion is an organic acid or base that stabilizes the siNA and polynucleotide delivery-enhancing polypeptide complex in solution.

The compositions and methods of the invention are useful as therapeutic tools to regulate expression of tumor necrosis factor-α (TNF-α) to treat or prevent symptoms of rheumatoid arthritis (RA). In this context the invention further provides compounds, compositions, and methods useful for modulating expression and activity of TNF-α by RNA interference (RNAi) using the short interfering RNA molecule LC20. LC20 is a double stranded 21-mer siRNA molecule with sequence homology to the human TNF-α gene. The LC20 nucleotide sequence is as follows:

GGGUCGGAACCCAAGUUATT
ATCCCAGCCUUGGGUUCGAAU

In more detailed embodiments, the invention provides a short interfering nucleic acid (siNA), short interfering RNA (siRNA), double-stranded RNA (dsRNA), micro-RNA (mRNA), and short hairpin RNA (shRNA) molecules, and related methods, that are effective for modulating expression of TNF-α and/or TNF-α genes to prevent or alleviate symptoms of RA in mammalian subjects. Within these and related therapeutic compositions and methods, the use of chemically-modified siNAs will often improve properties of the modified siNAs in comparison to properties of native siNA molecules, for example by providing increased resistance to nuclease degradation in vivo, and/or through improved cellular uptake. As can be readily determined according to the disclosure herein, useful siNAs having multiple chemical modifications will retain their RNAi activity. The siNA molecules of the instant invention thus provide useful reagents and methods for a variety of therapeutic, diagnostic, target validation, genomic discovery, genetic engineering, and pharmacogenomic applications.

Administration

This siNAs of the present invention may be administered in any form, for example transdermally or by local injection (e.g., local injection at sites of psoriatic plaques to treat psoriasis, or into the joints of patients afflicted with psoriatic arthritis or RA). In more detailed embodiments, the invention provides formulations and methods to administer therapeutically effective amounts of siNAs directed against of a mRNA of TNF-α, which effectively down-regulate the TNF-α RNA and thereby reduce or prevent one or more TNF-α-associated inflammatory condition(s). Comparable methods and compositions are provided that target expression of one or more different genes associated with a selected disease condition in animal subjects, including any of a large number of genes whose expression is known to be aberrantly increased as a causal or contributing factor associated with the selected disease condition.

The siNA/polynucleotide delivery-enhancing polypeptide mixtures of the invention can be administered in conjunction with other standard treatments for a targeted disease condition, for example in conjunction with therapeutic agents effective against inflammatory diseases, such as RA or psoriasis. Examples of combinatorially useful and effective agents in this context include non-steroidal antiinflammatory drugs (NSAIDs), methotrexate, gold compounds, D-penicillamine, the antimalarials, sulfasalazine, glucocorticoids, and other TNF-α neutralizing agents such as infliximab and entracept.

Negatively charged polynucleotides of the invention (e.g., RNA or DNA) can be administered to a patient by any standard means, with or without stabilizers, buffers, and the like, to form a pharmaceutical composition. When it is desired to use a liposome delivery mechanism, standard protocols for formation of liposomes can be followed. The compositions of the present invention may also be formulated and used as tablets, capsules or elixirs for oral administration, suppositories for rectal administration, sterile solutions, suspensions for injectable administration, and the other compositions known in the art.

The present invention also includes pharmaceutically acceptable formulations of the compositions described herein. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or patient, including for example a human. Suitable forms, in part, depend upon the use or the route of entry, for example oral, transdermal, or by injection. Such forms should not prevent the composition or formulation from reaching a target cell (i.e., a cell to which the negatively charged nucleic acid is desirable for delivery). For example, pharmacological compositions injected into the blood stream should be soluble. Other factors are known in the art, and include considerations such as toxicity.

In exemplary embodiments, the instant invention features compositions comprising a small nucleic acid molecule, such as short interfering nucleic acid (siNA), a short interfering RNA (siRNA), a double-stranded RNA (dsRNA), micro-RNA (mRNA), or a short hairpin RNA (shRNA), admixed or complexed with, or conjugated to, a polynucleotide delivery-enhancing polypeptide.

As used herein, the term “short interfering nucleic acid”, “siNA”, “short interfering RNA”, “siRNA”, “short interfering nucleic acid molecule”, “short interfering oligonucleotide molecule”, or “chemically-modified short interfering nucleic acid molecule”, refers to any nucleic acid molecule capable of inhibiting or down regulating gene expression or viral replication, for example by mediating RNA interference “RNAi” or gene silencing in a sequence-specific manner. Within exemplary embodiments, the siNA is a double-stranded polynucleotide molecule comprising self-complementary sense and antisense regions, wherein the antisense region comprises a nucleotide sequence that is complementary to a nucleotide sequence in a target nucleic acid molecule for down regulating expression, or a portion thereof, and the sense region comprises a nucleotide sequence corresponding to (i.e., which is substantially identical in sequence to) the target nucleic acid sequence or portion thereof.

“siNA” means a small interfering nucleic acid, for example a siRNA, that is a short-length double-stranded nucleic acid (or optionally a longer precursor thereof), and which is not unacceptably toxic in target cells. The length of useful siNAs within the invention will in certain embodiments be optimized at a length of approximately 20 to 50 bp long. However, there is no particular limitation in the length of useful siNAs, including siRNAs. For example, siNAs can initially be presented to cells in a precursor form that is substantially different than a final or processed form of the siNA that will exist and exert gene silencing activity upon delivery, or after delivery, to the target cell. Precursor forms of siNAs may, for example, include precursor sequence elements that are processed, degraded, altered, or cleaved at or following the time of delivery to yield a siNA that is active within the cell to mediate gene silencing. Thus, in certain embodiments, useful siNAs within the invention will have a precursor length, for example, of approximately 100-200 base pairs, 50-100 base pairs, or less than about 50 base pairs, which will yield an active, processed siNA within the target cell. In other embodiments, a useful siNA or siNA precursor will be approximately 10 to 49 bp, 15 to 35 bp, or about 21 to 30 bp in length.

In certain embodiments of the invention, as noted above, polynucleotide delivery-enhancing polypeptides are used to facilitate delivery of larger nucleic acid molecules than conventional siNAs, including large nucleic acid precursors of siNAs. For example, the methods and compositions herein may be employed for enhancing delivery of larger nucleic acids that represent “precursors” to desired siNAs, wherein the precursor amino acids may be cleaved or otherwise processed before, during or after delivery to a target cell to form an active siNA for modulating gene expression within the target cell. For example, a siNA precursor polynucleotide may be selected as a circular, single-stranded polynucleotide, having two or more loop structures and a stem comprising self-complementary sense and antisense regions, wherein the antisense region comprises a nucleotide sequence that is complementary to a nucleotide sequence in a target nucleic acid molecule or a portion thereof, and the sense region having nucleotide sequence corresponding to the target nucleic acid sequence or a portion thereof, and wherein the circular polynucleotide can be processed either in vivo or in vitro to generate an active siNA molecule capable of mediating RNAi.

In mammalian cells, dsRNAs longer than 30 base pairs can activate the dsRNA-dependent kinase PKR and 2′-5′-oligoadenylate synthetase, normally induced by interferon. The activated PKR inhibits general translation by phosphorylation of the translation factor eukaryotic initiation factor 2α (eIF2α), while 2′-5′-oligoadenylate synthetase causes nonspecific mRNA degradation via activation of RNase L. By virtue of their small size (referring particularly to non-precursor forms), usually less than 30 base pairs, and most commonly between about 17-19, 19-21, or 21-23 base pairs, the siNAs of the present invention avoid activation of the interferon response.

In contrast to the nonspecific effect of long dsRNA, siRNA can mediate selective gene silencing in the mammalian system. Hairpin RNAs, with a short loop and 19 to 27 base pairs in the stem, also selectively silence expression of genes that are homologous to the sequence in the double-stranded stem. Mammalian cells can convert short hairpin RNA into siRNA to mediate selective gene silencing.

RISC mediates cleavage of single stranded RNA having sequence complementary to the antisense strand of the siRNA duplex. Cleavage of the target RNA takes place in the middle of the region complementary to the antisense strand of the siRNA duplex. Studies have shown that 21 nucleotide siRNA duplexes are most active when containing two nucleotide 3′-overhangs. Furthermore, complete substitution of one or both siRNA strands with 2′-deoxy (2′-H) or 2′-O-methyl nucleotides abolishes RNAi activity, whereas substitution of the 3′-terminal siRNA overhang nucleotides with deoxy nucleotides (2′-H) has been reported to be tolerated.

Studies have shown that replacing the 3′-overhanging segments of a 21-mer siRNA duplex having 2 nucleotide 3′ overhangs with deoxyribonucleotides does not have an adverse effect on RNAi activity. Replacing up to 4 nucleotides on each end of the siRNA with deoxyribonucleotides has been reported to be well tolerated whereas complete substitution with deoxyribonucleotides results in no RNAi activity.

Alternatively, the siNAs can be delivered as single or multiple transcription products expressed by a polynucleotide vector encoding the single or multiple siNAs and directing their expression within target cells. In these embodiments the double-stranded portion of a final transcription product of the siRNAs to be expressed within the target cell can be, for example, 15 to 49 bp, 15 to 35 bp, or about 21 to 30 bp long. Within exemplary embodiments, double-stranded portions of siNAs, in which two strands pair up, are not limited to completely paired nucleotide segments, and may contain nonpairing portions due to mismatch (the corresponding nucleotides are not complementary), bulge (lacking in the corresponding complementary nucleotide on one strand), overhang, and the like. Nonpairing portions can be contained to the extent that they do not interfere with siNA formation. In more detailed embodiments, a “bulge” may comprise 1 to 2 nonpairing nucleotides, and the double-stranded region of siNAs in which two strands pair up may contain from about 1 to 7, or about 1 to 5 bulges. In addition, “mismatch” portions contained in the double-stranded region of siNAs may be present in numbers from about 1 to 7, or about 1 to 5. Most often in the case of mismatches, one of the nucleotides is guanine, and the other is uracil. Such mismatching may be attributable, for example, to a mutation from C to T, G to A, or mixtures thereof, in a corresponding DNA coding for sense RNA, but other cause are also contemplated. Furthermore, in the present invention the double-stranded region of siNAs in which two strands pair up may contain both bulge and mismatched portions in the approximate numerical ranges specified.

The terminal structure of siNAs of the invention may be either blunt or cohesive (overhanging) as long as the siNA retains its activity to silence expression of target genes. The cohesive (overhanging) end structure is not limited only to the 3′ overhang as reported by others. On the contrary, the 5′ overhanging structure may be included as long as it is capable of inducing a gene silencing effect such as by RNAi. In addition, the number of overhanging nucleotides is not limited to reported limits of 2 or 3 nucleotides, but can be any number as long as the overhang does not impair gene silencing activity of the siNA. For example, overhangs may comprise from about 1 to 8 nucleotides, more often from about 2 to 4 nucleotides. The total length of siNAs having cohesive end structure is expressed as the sum of the length of the paired double-stranded portion and that of a pair comprising overhanging single-strands at both ends. For example, in the exemplary case of a 19 bp double-stranded RNA with 4 nucleotide overhangs at both ends, the total length is expressed as 23 bp. Furthermore, since the overhanging sequence may have low specificity to a target gene, it is not necessarily complementary (antisense) or identical (sense) to the target gene sequence. Furthermore, as long as the siNA is able to maintain its gene silencing effect on the target gene, it may contain low molecular weight structure (for example a natural RNA molecule such as tRNA, rRNA or viral RNA, or an artificial RNA molecule), for example, in the overhanging portion at one end.

In addition, the terminal structure of the siNAs may have a stem-loop structure in which ends of one side of the double-stranded nucleic acid are connected by a linker nucleic acid, e.g., a linker RNA. The length of the double-stranded region (stem-loop portion) can be, for example, 15 to 49 bp, often 15 to 35 bp, and more commonly about 21 to 30 bp long. Alternatively, the length of the double-stranded region that is a final transcription product of siNAs to be expressed in a target cell may be, for example, approximately 15 to 49 bp, 15 to 35 bp, or about 21 to 30 bp long. When linker segments are employed, there is no particular limitation in the length of the linker as long as it does not hinder pairing of the stem portion. For example, for stable pairing of the stem portion and suppression of recombination between DNAs coding for this portion, the linker portion may have a clover-leaf tRNA structure. Even if the linker has a length that would hinder pairing of the stem portion, it is possible, for example, to construct the linker portion to include introns so that the introns are excised during processing of a precursor RNA into mature RNA, thereby allowing pairing of the stem portion. In the case of a stem-loop siRNA, either end (head or tail) of RNA with no loop structure may have a low molecular weight RNA. As described above, these low molecular weight RNAs may include a natural RNA molecule, such as tRNA, rRNA or viral RNA, or an artificial RNA molecule.

The siNA can also comprise a single stranded polynucleotide having nucleotide sequence complementary to nucleotide sequence in a target nucleic acid molecule or a portion thereof (for example, where such siNA molecule does not require the presence within the siNA molecule of nucleotide sequence corresponding to the target nucleic acid sequence or a portion thereof), wherein the single stranded polynucleotide can further comprise a terminal phosphate group, such as a 5′-phosphate (see for example Martinez et al., Cell. 110:563-574, 2002, and Schwarz et al., Molecular Cell 10:537-568, 2002, or 5′,3′-diphosphate.

As used herein, the term siNA molecule is not limited to molecules containing only naturally-occurring RNA or DNA, but also encompasses chemically-modified nucleotides and non-nucleotides. In certain embodiments, the short interfering nucleic acid molecules of the invention lack 2′-hydroxy (2′-OH) containing nucleotides. In certain embodiments short interfering nucleic acids do not require the presence of nucleotides having a 2′-hydroxy group for mediating RNAi and as such, short interfering nucleic acid molecules of the invention optionally do not include any ribonucleotides (e.g., nucleotides having a 2′-OH group). Such siNA molecules that do not require the presence of ribonucleotides within the siNA molecule to support RNAi can however have an attached linker or linkers or other attached or associated groups, moieties, or chains containing one or more nucleotides with 2′-OH groups. Optionally, siNA molecules can comprise ribonucleotides at about 5, 10, 20, 30, 40, or 50% of the nucleotide positions.

As used herein, the term siNA is meant to be equivalent to other terms used to describe nucleic acid molecules that are capable of mediating sequence specific RNAi, for example short interfering RNA (siRNA), double-stranded RNA (dsRNA), micro-RNA (mRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others.

In other embodiments, siNA molecules for use within the invention may comprise separate sense and antisense sequences or regions, wherein the sense and antisense regions are covalently linked by nucleotide or non-nucleotide linker molecules, or are alternately non-covalently linked by ionic interactions, hydrogen bonding, van der waals interactions, hydrophobic intercations, and/or stacking interactions.

“Antisense RNA” is an RNA strand having a sequence complementary to a target gene mRNA, and thought to induce RNAi by binding to the target gene mRNA. “Sense RNA” has a sequence complementary to the antisense RNA, and annealed to its complementary antisense RNA to form siRNA. These antisense and sense RNAs have been conventionally synthesized with an RNA synthesizer.

As used herein, the term “RNAi construct” is a generic term used throughout the specification to include small interfering RNAs (siRNAs), hairpin RNAs, and other RNA species which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo. Optionally, the siRNA include single strands or double strands of siRNA.

An siHybrid molecule is a double-stranded nucleic acid that has a similar function to siRNA. Instead of a double-stranded RNA molecule, an siHybrid is comprised of an RNA strand and a DNA strand. Preferably, the RNA strand is the antisense strand as that is the strand that binds to the target mRNA. The siHybrid created by the hybridization of the DNA and RNA strands have a hybridized complementary portion and preferably at least one 3′ overhanging end.

siNAs for use within the invention can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure, for example wherein the double stranded region is about 19 base pairs). The antisense strand may comprise a nucleotide sequence that is complementary to a nucleotide sequence in a target nucleic acid molecule or a portion thereof, and the sense strand may comprise a nucleotide sequence corresponding to the target nucleic acid sequence or a portion thereof. Alternatively, the siNA can be assembled from a single oligonucleotide, where the self-complementary sense and antisense regions of the siNA are linked by means of a nucleic acid-based or non-nucleic acid-based linker(s).

Within additional embodiments, siNAs for intracellular delivery according to the methods and compositions of the invention can be a polynucleotide with a duplex, asymmetric duplex, hairpin or asymmetric hairpin secondary structure, having self-complementary sense and antisense regions, wherein the antisense region comprises a nucleotide sequence that is complementary to a nucleotide sequence in a separate target nucleic acid molecule or a portion thereof, and the sense region comprises a nucleotide sequence corresponding to the target nucleic acid sequence or a portion thereof.

Non-limiting examples of chemical modifications that can be made in an siNA include without limitation phosphorothioate internucleotide linkages, 2′-deoxyribonucleotides, 2′-O-methyl ribonucleotides, 2′-deoxy-2′-fluoro ribonucleotides, “universal base” nucleotides, “acyclic” nucleotides, 5-C-methyl nucleotides, and terminal glyceryl and/or inverted deoxy abasic residue incorporation. These chemical modifications, when used in various siNA constructs, are shown to preserve RNAi activity in cells while at the same time, dramatically increasing the serum stability of these compounds.

In a non-limiting example, the introduction of chemically-modified nucleotides into nucleic acid molecules provides a powerful tool in overcoming potential limitations of in vivo stability and bioavailability inherent to native RNA molecules that are delivered exogenously. For example, the use of chemically-modified nucleic acid molecules can enable a lower dose of a particular nucleic acid molecule for a given therapeutic effect since chemically-modified nucleic acid molecules tend to have a longer half-life in serum. Furthermore, certain chemical modifications can improve the bioavailability of nucleic acid molecules by targeting particular cells or tissues and/or improving cellular uptake of the nucleic acid molecule. Therefore, even if the activity of a chemically-modified nucleic acid molecule is reduced as compared to a native nucleic acid molecule, for example, when compared to an all-RNA nucleic acid molecule, the overall activity of the modified nucleic acid molecule can be greater than that of the native molecule due to improved stability and/or delivery of the molecule. Unlike native unmodified siNA, chemically-modified siNA can also minimize the possibility of activating interferon activity in humans.

The siNA molecules described herein, the antisense region of a siNA molecule of the invention can comprise a phosphorothioate internucleotide linkage at the 3′-end of said antisense region. In any of the embodiments of siNA molecules described herein, the antisense region can comprise about one to about five phosphorothioate internucleotide linkages at the 5′-end of said antisense region. In any of the embodiments of siNA molecules described herein, the 3′-terminal nucleotide overhangs of a siNA molecule of the invention can comprise ribonucleotides or deoxyribonucleotides that are chemically-modified at a nucleic acid sugar, base, or backbone. In any of the embodiments of siNA molecules described herein, the 3′-terminal nucleotide overhangs can comprise one or more universal base ribonucleotides. In any of the embodiments of siNA molecules described herein, the 3′-terminal nucleotide overhangs can comprise one or more acyclic nucleotides.

For example, in a non-limiting example, the invention features a chemically-modified short interfering nucleic acid (siNA) having about 1, 2, 3, 4, 5, 6, 7, 8 or more phosphorothioate internucleotide linkages in one siNA strand. In yet another embodiment, the invention features a chemically-modified short interfering nucleic acid (siNA) individually having about 1, 2, 3, 4, 5, 6, 7, 8 or more phosphorothioate internucleotide linkages in both siNA strands. The phosphorothioate internucleotide linkages can be present in one or both oligonucleotide strands of the siNA duplex, for example in the sense strand, the antisense strand, or both strands. The siNA molecules of the invention can comprise one or more phosphorothioate internucleotide linkages at the 3′-end, the 5′-end, or both of the 3′- and 5′-ends of the sense strand, the antisense strand, or both strands. For example, an exemplary siNA molecule of the invention can comprise about 1 to about 5 or more (e.g., about 1, 2, 3, 4, 5, or more) consecutive phosphorothioate internucleotide linkages at the 5′-end of the sense strand, the antisense strand, or both strands. In another non-limiting example, an exemplary siNA molecule of the invention can comprise one or more (e.g., about 1, 2, 3, 4, 5, 6, 7, 8, 9, 10, or more) pyrimidine phosphorothioate internucleotide linkages in the sense strand, the antisense strand, or both strands. In yet another non-limiting example, an exemplary siNA molecule of the invention can comprise one or more (e.g., about 1, 2, 3, 4, 5, 6, 7, 8, 9, 10, or more) purine phosphorothioate internucleotide linkages in the sense strand, the antisense strand, or both strands.

An siNA molecule may be comprised of a circular nucleic acid molecule, wherein the siNA is about 38 to about 70 (e.g., about 38, 40, 45, 50, 55, 60, 65, or 70) nucleotides in length having about 18 to about 23 (e.g., about 18, 19, 20, 21, 22, or 23) base pairs wherein the circular oligonucleotide forms a dumbbell shaped structure having about 19 base pairs and 2 loops.

A circular siNA molecule contains two loop motifs, wherein one or both loop portions of the siNA molecule is biodegradable. For example, a circular siNA molecule of the invention is designed such that degradation of the loop portions of the siNA molecule in vivo can generate a double-stranded siNA molecule with 3′-terminal overhangs, such as 3′-terminal nucleotide overhangs comprising about 2 nucleotides.

Modified nucleotides present in siNA molecules, preferably in the antisense strand of the siNA molecules, but also optionally in the sense and/or both antisense and sense strands, comprise modified nucleotides having properties or characteristics similar to naturally occurring ribonucleotides. For example, the invention features siNA molecules including modified nucleotides having a Northern conformation (e.g., Northern pseudorotation cycle, see for example, Saenger, Principles of Nucleic Acid Structure, Springer-Verlag ed., 1984). As such, chemically modified nucleotides present in the siNA molecules of the invention, preferably in the antisense strand of the siNA molecules of the invention, but also optionally in the sense and/or both antisense and sense strands, are resistant to nuclease degradation while at the same time maintaining the capacity to mediate RNAi. Non-limiting examples of nucleotides having a northern configuration include locked nucleic acid (LNA) nucleotides (e.g., 2′-O, 4′-C-methylene-(D-ribofuranosyl) nucleotides); 2′-methoxyethoxy (MOE) nucleotides; 2′-methyl-thio-ethyl, 2′-deoxy-2′-fluoro micleotides. 2′-deoxy-2′-chloro nucleotides, 2′-azido nucleotides, and 2′-O-methyl nucleotides.

The sense strand of a double stranded siNA molecule may have a terminal cap moiety such as an inverted deoxyabasic moiety, at the 3′-end, 5′-end, or both 3′and 5′-ends of the sense strand.

Non-limiting examples of conjugates include conjugates and ligands described in Vargeese et al., U.S. application Ser. No. 10/427,160, filed Apr. 30, 2003, incorporated by reference herein in its entirety, including the drawings. In another embodiment, the conjugate is covalently attached to the chemically-modified siNA molecule via a biodegradable linker. In one embodiment, the conjugate molecule is attached at the 3′-end of either the sense strand, the antisense strand, or both strands of the chemically-modified siNA molecule. In another embodiment, the conjugate molecule is attached at the 5′-end of either the sense strand, the antisense strand, or both strands of the chemically-modified siNA molecule. In yet another embodiment, the conjugate molecule is attached both the 3′-end and 5′-end of either the sense strand, the antisense strand, or both strands of the chemically-modified siNA molecule, or any combination thereof. In one embodiment, a conjugate molecule of the invention comprises a molecule that facilitates delivery of a chemically-modified siNA molecule into a biological system, such as a cell. In another embodiment, the conjugate molecule attached to the chemically-modified siNA molecule is a poly ethylene glycol, human serum albumin, or a ligand for a cellular receptor that can mediate cellular uptake. Examples of specific conjugate molecules contemplated by the instant invention that can be attached to chemically-modified siNA molecules are described in Vargeese et al., U.S. Patent Application Publication No. 20030130186, published Jul. 10, 2003, and U.S. Patent Application Publication No. 20040110296, published Jun. 10, 2004. The type of conjugates used and the extent of conjugation of siNA molecules of the invention can be evaluated for improved pharmacokinetic profiles, bioavailability, and/or stability of siNA constructs while at the same time maintaining the ability of the siNA to mediate RNAi activity. As such, one skilled in the art can screen siNA constructs that are modified with various conjugates to determine whether the siNA conjugate complex possesses improved properties while maintaining the ability to mediate RNAi, for example in animal models as are generally known in the art.

A siNA further may be further comprised of a nucleotide, non-nucleotide, or mixed nucleotide/non-nucleotide linker that joins the sense region of the siNA to the antisense region of the siNA. In one embodiment, a nucleotide linker can be a linker of >2 nucleotides in length, for example about 3, 4, 5, 6, 7, 8, 9, or 10 nucleotides in length. In another embodiment, the nucleotide linker can be a nucleic acid aptamer. By “aptamer” or “nucleic acid aptamer” as used herein is meant a nucleic acid molecule that binds specifically to a target molecule wherein the nucleic acid molecule has sequence that comprises a sequence recognized by the target molecule in its natural setting. Alternately, an aptamer can be a nucleic acid molecule that binds to a target molecule where the target molecule does not naturally bind to a nucleic acid. The target molecule can be any molecule of interest. For example, the aptamer can be used to bind to a ligand-binding domain of a protein, thereby preventing interaction of the naturally occurring ligand with the protein. This is a non-limiting example and those in the art will recognize that other embodiments can be readily generated using techniques generally known in the art. See, for example, Gold et al, Annu. Rev. Biochem. 64:763, 1995; Brody and Gold, J. Biotechnol. 74:5, 2000; Sun, Curr. Opin. Mol Ther. 2:100, 2000; Kusser, J. Biotechnol. 74:27, 2000; Hermann and Patel, Science 287:820, 2000; and Jayasena, Clinical Chemistry 45:1628, 1999.

A non-nucleotide linker may be comprised of an abasic nucleotide, polyether, polyamine, polyamide, peptide, carbohydrate, lipid, polyhydrocarbon, or other polymeric compounds (e.g. polyethylene glycols such as those having between 2 and 100 ethylene glycol units). Specific examples include those described by Seela and Kaiser, Nucleic Acids Res. 18:6353, 1990 and Nucleic Acids Res. 15:3113, 1987; Cload and Schepartz, J. Am. Chem. Soc. 113:6324, 1991; Richardson and Schepartz, J. Am. Chem. Soc. 113:5109, 1991; Ma et al., Nucleic Acids Res. 21:2585, 1993 and Biochemistry 32:1751, 1993; Durand et al., Nucleic Acids Res. 18:6353, 1990; McCurdy et al., Nucleosides & Nucleotides 10:287, 1991; Jschke et al., Tetrahedron Lett. 34:301, 1993; Ono et al., Biochemistry 30:9914, 1991; Arnold et al., International Publication No. WO 89/02439; Usman et al., International Publication No. WO 95/06731; Dudycz et al., International Publication No. WO 95/11910 and Ferentz and Verdine, J. Am. Chem. Soc. 113:4000, 1991. A “non-nucleotide” further means any group or compound that can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thyrnine, for example at the C1 position of the sugar.

In one embodiment, the invention features modified siNA molecules, with phosphate backbone modifications comprising one or more phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications, see Hunziker and Leumann, “Nucleic Acid Analogues: Synthesis and Properties, in Modern Synthetic Methods,” VCH, 331-417, 1995, and Mesmaeker et al., “Novel Backbone Replacements for Oligonucleotides, in Carbohydrate Modifications in Antisense Research,” ACS, 24-39, 1994.

Synthesis of siNA:

The synthesis of a siNA molecule of the invention, which can be chemically-modified, comprises: (a) synthesis of two complementary strands of the siNA molecule; (b) annealing the two complementary strands together under conditions suitable to obtain a double-stranded siNA molecule. In another embodiment, synthesis of the two complementary strands of the siNA molecule is by solid phase oligonucleotide synthesis. In yet another embodiment, synthesis of the two complementary strands of the siNA molecule is by solid phase tandem oligonucleotide synthesis.

Oligonucleotides (e.g., certain modified oligonucleotides or portions of oligonucleotides lacking ribonucleotides) are synthesized using protocols known in the art, for example as described in Caruthers et al., Methods in Enzymology 211:3-19, 1992; Thompson et al., International PCT Publication No. WO 99/54459; Wincott et al., Nucleic Acids Res. 23:2677-2684, 1995; Wincott et al., Methods Mol. Bio. 74:59, 1997; Brennan et al., Biotechnol Bioeng. 61:33-45, 1998, and Brennan, U.S. Pat. No. 6,001,311. Synthesis of RNA, including certain siNA molecules of the invention, follows general procedures as described, for example, in Usman et al., J. Am. Chem. Soc. 109:7845, 1987; Scaringe et al., Nucleic Acids Res. 18:5433, 1990; and Wincott et al., Nucleic Acids Res. 23:2677-2684, 1995; Wincott et al., Methods Mol. Bio. 74:59, 1997.

Supplemental or complementary methods for delivery of nucleic acid molecules for use within then invention are described, for example, in Akhtar et al., Trends Cell Bio. 2:139, 1992; “Delivery Strategies for Antisense Oligonucleotide Therapeutics,” ed. Akhtar, 1995, Maurer et al., Mol. Membr. Biol. 16:129-140, 1999; Hofland and Huang, Handb. Exp. Pharmacol. 137:165-192, 1999; and Lee et al., ACS Symp. Ser. 752:184-192, 2000. Sullivan et al., International PCT Publication No. WO 94/02595, further describes general methods for delivery of enzymatic nucleic acid molecules. These protocols can be utilized to supplement or complement delivery of virtually any nucleic acid molecule contemplated within the invention.

Delivery Methods:

Nucleic acid molecules and polynucleotide delivery-enhancing polypeptides can be administered to cells by a variety of methods known to those of skill in the art, including, but not restricted to, administration within formulations that comprise the siNA and polynucleotide delivery-enhancing polypeptide alone, or that further comprise one or more additional components, such as a pharmaceutically acceptable carrier, diluent, excipient, adjuvant, emulsifier, buffer, stabilizer, preservative, and the like. In certain embodiments, the siNA and/or the polynucleotide delivery-enhancing polypeptide can be encapsulated in liposomes, administered by iontophoresis, or incorporated into other vehicles, such as hydrogels, cyclodextrins, biodegradable nanocapsules, bioadhesive microspheres, or proteinaceous vectors (see e.g., O'Hare and Normand, International PCT Publication No. WO 00/53722). Alternatively, a nucleic acid/peptide/vehicle combination can be locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry et al., Clin. Cancer Res. 5:2330-2337, 1999 and Barry et al., International PCT Publication No. WO 99/31262.

Methods for the delivery of nucleic acid molecules are described in Akhtar et al., Trends Cell Bio. 2:139, 1992; “Delivery Strategies for Antisense Oligonucleotide Therapeutics,” ed. Akhtar, 1995; Maurer et al., Mol. Membr. Biol. 16:129-140, 1999; Hofland and Huang, Handb. Exp. Pharmacol. 137:165-192, 1999; and Lee et al., ACS Symp. Ser. 752:184-192, 2000. Beigelman et al., U.S. Pat. No. 6,395,713 and Sullivan et al., PCT WO 94/02595 further describe the general methods for delivery of nucleic acid molecules. These protocols can be utilized for the delivery of virtually any nucleic acid molecule. Nucleic acid molecules can be administered to cells by a variety of methods known to those of skill in the art, including, but not restricted to, encapsulation in liposomes, by iontophoresis, or by incorporation into other vehicles, such as biodegradable polymers, hydrogels, cyclodextrins (see for example, Gonzalez et al., Bioconjugate Chem. 10:1068-1074, 1999; Wang et al., International PCT Publication Nos. WO 03/47518 and WO 03/46185), poly(lactic-co-glycolic)ac-id (PLGA) and PLCA microspheres (see for example, U.S. Pat. No. 6,447,796 and U.S. Patent Application Publication No. US 2002130430), biodegradable nanocapsules, and bioadhesive microspheres, or by proteinaceous vectors (O'Hare and Normand, International PCT Publication No. WO 00/53722). Alternatively, the nucleic acid/vehicle combination is locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry et al., Clin. Cancer Res. 5:2330-2337, 1999, and Barry et al., International PCT Publication No. WO 99/31262. The molecules of the instant invention can be used as pharmaceutical agents. Pharmaceutical agents prevent, modulate the occurrence, or treat (alleviate a symptom to some extent, preferably all of the symptoms) of a disease state in a subject.

Terms Defined

The term “ligand” refers to any compound or molecule, such as a drug, peptide, hormone, or neurotransmitter, that is capable of interacting with another compound, such as a receptor, either directly or indirectly. The receptor that interacts with a ligand can be present on the surface of a cell or can alternately be an intercellular receptor. Interaction of the ligand with the receptor can result in a biochemical reaction, or can simply be a physical interaction or association.

By “asymmetric hairpin” as used herein is meant a linear siNA molecule comprising an antisense region, a loop portion that can comprise nucleotides or non-nucleotides, and a sense region that comprises fewer nucleotides than the antisense region to the extent that the sense region has enough complementary nucleotides to base pair with the antisense region and form a duplex with loop. For example, an asymmetric hairpin siNA molecule of the invention can comprise an antisense region having length sufficient to mediate RNAi in a T-cell (e.g., about 19 to about 22 (e.g., about 19, 20, 21, or 22) nucleotides) and a loop region comprising about 4 to about 8 (e.g., about 4, 5, 6, 7, or 8) nucleotides, and a sense region having about 3 to about 18 (e.g., about 3, 4, 5, 6, 7, 8, 9, 10, 11, 12, 13, 14, 15, 16, 17, or 18) nucleotides that are complementary to the antisense region. The asymmetric hairpin siNA molecule can also comprise a 5′-terminal phosphate group that can be chemically modified. The loop portion of the asymmetric hairpin siNA molecule can comprise nucleotides, non-nucleotides, linker molecules, or conjugate molecules as described herein.

By “asymmetric duplex” as used herein is meant a siNA molecule having two separate strands comprising a sense region and an antisense region, wherein the sense region comprises fewer nucleotides than the antisense region to the extent that the sense region has enough complementary nucleotides to base pair with the antisense region and form a duplex. For example, an asymmetric duplex siNA molecule of the invention can comprise an antisense region having length sufficient to mediate RNAi in a T-cell (e.g., about 19 to about 22 (e.g., about 19, 20, 21, or 22) nucleotides) and a sense region having about 3 to about 18 (e.g., about 3, 4, 5, 6, 7, 8, 9, 10, 11, 12, 13, 14, 15, 16, 17, or 18) nucleotides that are complementary to the antisense region.

By “modulate gene expression” is meant that the expression of a target gene is upregulated or downregulated, which can include upregulation or downregulation of mRNA levels present in a cell, or of mRNA translation, or of synthesis of protein or protein subunits, encoded by the target gene. Modulation of gene expression can be determined also be the presence, quantity, or activity of one or more proteins or protein subunits encoded by the target gene that is up regulated or down regulated, such that expression, level, or activity of the subject protein or subunit is greater than or less than that which is observed in the absence of the modulator (e.g., a siRNA). For example, the term “modulate” can mean “inhibit,” but the use of the word “modulate” is not limited to this definition.

By “inhibit”, “down-regulate”, or “reduce” expression, it is meant that the expression of the gene, or level of RNA molecules or equivalent RNA molecules encoding one or more proteins or protein subunits, or level or activity of one or more proteins or protein subunits encoded by a target gene, is reduced below that observed in the absence of the nucleic acid molecules (e.g., siNA) of the invention. In one embodiment, inhibition, down-regulation or reduction with an siNA molecule is below that level observed in the presence of an inactive or attenuated molecule. In another embodiment, inhibition, down-regulation, or reduction with siNA molecules is below that level observed in the presence of, for example, an siNA molecule with scrambled sequence or with mismatches. In another embodiment, inhibition, down-regulation, or reduction of gene expression with a nucleic acid molecule of the instant invention is greater in the presence of the nucleic acid molecule than in its absence.

Gene “silencing” refers to partial or complete loss-of-function through targeted inhibition of gene expression in a cell and may also be referred to as “knock down.” Depending on the circumstances and the biological problem to be addressed, it may be preferable to partially reduce gene expression. Alternatively, it might be desirable to reduce gene expression as much as possible. The extent of silencing may be determined by methods known in the art, some of which are summarized in International Publication No. WO 99/32619. Depending on the assay, quantification of gene expression permits detection of various amounts of inhibition that may be desired in certain embodiments of the invention, including prophylactic and therapeutic methods, which will be capable of knocking down target gene expression, in terms of mRNA levels or protein levels or activity, for example, by equal to or greater than 10%, 30%, 50%, 75% 90%, 95% or 99% of baseline (i.e., normal) or other control levels, including elevated expression levels as may be associated with particular disease states or other conditions targeted for therapy.

The phrase “inhibiting expression of a target gene” refers to the ability of a siNA of the invention to initiate gene silencing of the target gene. To examine the extent of gene silencing, samples or assays of the organism of interest or cells in culture expressing a particular construct are compared to control samples lacking expression of the construct. Control samples (lacking construct expression) are assigned a relative value of 100%. Inhibition of expression of a target gene is achieved when the test value relative to the control is about 90%, often 50%, and in certain embodiments 25-0%. Suitable assays include, e.g., examination of protein or mRNA levels using techniques known to those of skill in the art such as dot blots, northern blots, in situ hybridization, ELISA, immunoprecipitation, enzyme function, as well as phenotypic assays known to those of skill in the art.

By “subject” is meant an organism, tissue, or cell, which may include an organism as the subject or as a donor or recipient of explanted cells or the cells that are themselves subjects for siNA delivery. “Subject” therefore may refers to an organism, organ, tissue, or cell, including in vitro or ex vivo organ, tissue or cellular subjects, to which the nucleic acid molecules of the invention can be administered and enhanced by polynucleotide delivery-enhancing polypeptides described herein. Exemplary subjects include mammalian individuals or cells, for example human patients or cells.

As used herein “cell” is used in its usual biological sense, and does not refer to an entire multicellular organism, e.g., specifically does not refer to a human. The cell can be present in an organism, e.g., birds, plants and mammals such as humans, cows, sheep, apes, monkeys, swine, dogs, and cats. The cell can be prokaryotic (e.g., bacterial cell) or eukaryotic (e.g., mammalian or plant cell). The cell can be of somatic or germ line origin, totipotent or pluripotent, dividing or non-dividing. The cell can also be derived from or can comprise a gamete or embryo, a stem cell, or a fully differentiated cell.

By “comprising” is meant including, but not limited to, whatever follows the word “comprising.” Thus, use of the term “comprising” indicates that the listed elements are required or mandatory, but that other elements are optional and may or may not be present. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

By “RNA” is meant a molecule comprising at least one ribonucleotide residue. By “ribonucleotide” is meant a nucleotide with a hydroxyl group at the 2′ position of a beta-D-ribo-furanose moiety. The terms include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siNA or internally, for example at one or more nucleotides of the RNA. Nucleotides in the RNA molecules of the instant invention can also comprise non-standard nucleotides, such as non-naturally occurring nucleotides or chemically synthesized nucleotides or deoxynucleotides. These altered RNAs can be referred to as analogs or analogs of naturally-occurring RNA.

By “highly conserved sequence region” is meant, a nucleotide sequence of one or more regions in a target gene does not vary significantly from one generation to the other or from one biological system to the other.

By “sense region” is meant a nucleotide sequence of a siNA molecule having complementarity to an antisense region of the siNA molecule. In addition, the sense region of a siNA molecule can comprise a nucleic acid sequence having homology with a target nucleic acid sequence.

By “antisense region” is meant a nucleotide sequence of a siNA molecule having complementarity to a target nucleic acid sequence. In addition, the antisense region of a siNA molecule can optionally comprise a nucleic acid sequence having complementarity to a sense region of the siNA molecule.

By “target nucleic acid” is meant any nucleic acid sequence whose expression or activity is to be modulated. The target nucleic acid can be DNA or RNA.

By “complementarity” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al., CSH Symp. Quant. Biol. LII pp. 123-133, 1987; Frier et al., Proc. Nat. Acad. Sci. USA 83:9373-9377, 1986; Turner et al., J. Am. Chem. Soc. 109:3783-3785, 1987). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, or 10 nucleotides out of a total of 10 nucleotides in the first oligonuelcotide being based paired to a second nucleic acid sequence having 10 nucleotides represents 50%, 60%, 70%, 80%, 90%, and 100% complementary respectively). “Perfectly complementary” means that all the contiguous residues of a nucleic acid sequence will hydrogen bond with the same number of contiguous residues in a second nucleic acid sequence.

The term “universal base” as used herein refers to nucleotide base analogs that form base pairs with each of the natural DNA/RNA bases with little discrimination between them. Non-limiting examples of universal bases include C-phenyl, C-naphthyl and other aromatic derivatives, inosine, azole carboxamides, and nitroazole derivatives such as 3-nitropyrrole, 4-nitroindole, 5-nitroindole, and 6-nitroindole as known in the art (see for example, Loakes, Nucleic Acids Research 29:2437-2447, 2001).

The term “acyclic nucleotide” as used herein refers to any nucleotide having an acyclic ribose sugar, for example where any of the ribose carbons (C1, C2, C3, C4, or C5), are independently or in combination absent from the nucleotide.

The term “biodegradable” as used herein, refers to degradation in a biological system, for example enzymatic degradation or chemical degradation.

The term “biologically active molecule” as used herein, refers to compounds or molecules that are capable of eliciting or modifying a biological response in a system. Non-limiting examples of biologically active siNA molecules either alone or in combination with other molecules contemplated by the instant invention include therapeutically active molecules such as antibodies, cholesterol, hormones, antivirals, peptides, proteins, chemotherapeutics, small molecules, vitamins, co-factors, nucleosides, nucleotides, oligonucleotides, enzymatic nucleic acids, antisense nucleic acids, triplex forming oligonucleotides, 2,5-A chimeras, siNA, dsRNA, allozymes, aptamers, decoys and analogs thereof. Biologically active molecules of the invention also include molecules capable of modulating the pharmacokinetics and/or pharmacodynamics of other biologically active molecules, for example, lipids and polymers such as polyamines, polyamides, polyethylene glycol and other polyethers.

The term “phospholipid” as used herein, refers to a hydrophobic molecule comprising at least one phosphorus group. For example, a phospholipid can comprise a phosphorus-containing group and saturated or unsaturated alkyl group, optionally substituted with OH, COOH, oxo, amine, or substituted or unsubstituted aryl groups.

By “cap structure” is meant chemical modifications, which have been incorporated at either terminus of the oligonucleotide (see, for example, Adamic et al., U.S. Pat. No. 5,998,203, incorporated by reference herein). These terminal modifications protect the nucleic acid molecule from exonuclease degradation, and may help in delivery and/or localization within a cell. The cap may be present at the 5′-terminus (5′-cap) or at the 3′-terminal (3′-cap) or may be present on both termini. In non-limiting examples, the 5′-cap includes, but is not limited to, glyceryl, inverted deoxy abasic residue (moiety); 4′,5′-methylene nucleotide; 1-(beta-D-erythrofuranosyl) nucleotide, 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety.

Non-limiting examples of the 3′-cap include, but are not limited to, glyceryl, inverted deoxy abasic residue (moiety), 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Lyer, Tetrahedron 49:1925, 1993,; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine and therefore lacks a base at the 1′-position.

By “nucleotide” as used herein is as recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety (also referred to interchangeably as nucleotide analogs, modified nucleotides, non-natural nucleotides, non-standard nucleotides and other; see, for example, Usman and McSwiggen, supra; Eckstein et al., International PCT Publication No. WO 92/07065; Usman et al, International PCT Publication No. WO 93/15187; Uhlman & Peyman, supra, all are hereby incorporated by reference herein). There are several examples of modified nucleic acid bases known in the art as summarized by Limbach et al, Nucleic Acids Res. 22:2183, 1994. Some of the non-limiting examples of base modifications that can be introduced into nucleic acid molecules include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin et al., Biochemistry 35:14090, 1996; Uhlman & Peyman, supra). By “modified bases” in this aspect is meant nucleotide bases other than adenine, guanine, cytosine and uracil at 1′position or their equivalents.

By “target site” is meant a sequence within a target RNA that is “targeted” for cleavage mediated by a siNA construct which contains sequences within its antisense region that are complementary to the target sequence.

By “detectable level of cleavage” is meant cleavage of target RNA (and formation of cleaved product RNAs) to an extent sufficient to discern cleavage products above the background of RNAs produced by random degradation of the target RNA. Production of cleavage products from 1-5% of the target RNA is sufficient to detect above the background for most methods of detection.

By “biological system” is meant, material, in a purified or unpurified form, from biological sources, including but not limited to human, animal, plant, insect, bacterial, viral or other sources, wherein the system comprises the components required for RNAi acitivity. The term “biological system” includes, for example, a cell, tissue, or organism, or extract thereof. The term biological system also includes reconstituted RNAi systems that can be used in an in vitro setting.

The term “biodegradable linker” as used herein, refers to a nucleic acid or non-nucleic acid linker molecule that is designed as a biodegradable linker to connect one molecule to another molecule, for example, a biologically active molecule to a siNA molecule of the invention or the sense and antisense strands of a siNA molecule of the invention. The biodegradable linker is designed such that its stability can be modulated for a particular purpose, such as delivery to a particular tissue or cell type. The stability of a nucleic acid-based biodegradable linker molecule can be modulated by using various chemistries, for example combinations of ribonucleotides, deoxyribonucleotides, and chemically-modified nucleotides, such as 2′-O-methyl, 2′-fluoro, 2′-amino, 2′-O-amino, 2′-C-allyl, 2′-O-allyl, and other 2′-modified or base modified nucleotides. The biodegradable nucleic acid linker molecule can be a dimer, trimer, tetramer or longer nucleic acid molecule, for example, an oligonucleotide of about 2, 3, 4, 5, 6, 7, 8, 9, 10, 11, 12, 13, 14, 15, 16, 17, 18, 19, or 20 nucleotides in length, or can comprise a single nucleotide with a phosphorus-based linkage, for example, a phosphoramidate or phosphodiester linkage. The biodegradable nucleic acid linker molecule can also comprise nucleic acid backbone, nucleic acid sugar, or nucleic acid base modifications.

By “abasic” is meant sugar moieties lacking a base or having other chemical groups in place of a base at the 1′position, see for example, Adamic et al., U.S. Pat. No. 5,998,203.

By “unmodified nucleoside” is meant one of the bases adenine, cytosine, guanine, thymine, or uracil joined to the 1′carbon of .beta.-D-ribo-furanose.

By “modified nucleoside” is meant any nucleotide base which contains a modification in the chemical structure of an unmodified nucleotide base, sugar and/or phosphate. Non-limiting examples of modified nucleotides are shown by Formulae I-VII and/or other modifications described herein.

In connection with 2′-modified nucleotides as described for the present invention, by “amino” is meant 2′-NH2 or 2′-O—-NH2, which can be modified or unmodified. Such modified groups are described, for example, in Eckstein et al., U.S. Pat. No. 5,672,695 and Matulic-Adamic et al., U.S. Pat. No. 6,248,878.

The siNA molecules can be complexed with cationic lipids, packaged within liposomes, or otherwise delivered to target cells or tissues. The nucleic acid or nucleic acid complexes can be locally administered to through injection, infusion pump or stent, with or without their incorporation in biopolymers. In another embodiment, polyethylene glycol (PEG) can be covalently attached to siNA compounds of the present invention, to the polynucleotide delivery-enhancing polypeptide, or both. The attached PEG can be any molecular weight, preferably from about 2,000 to about 50,000 Daltons (Da).

The sense region can be connected to the antisense region via a linker molecule, such as a polynucleotide linker or a non-nucleotide linker.

“Inverted repeat” refers to a nucleic acid sequence comprising a sense and an antisense element positioned so that they are able to form a double stranded siRNA when the repeat is transcribed. The inverted repeat may optionally include a linker or a heterologous sequence such as a self-cleaving ribozyme between the two elements of the repeat. The elements of the inverted repeat have a length sufficient to form a double stranded RNA. Typically, each element of the inverted repeat is about 15 to about 100 nucleotides in length, preferably about 20-30 base nucleotides, preferably about 20-25 nucleotides in length, e.g., 20, 21, 22, 23, 24, 25, 26, 27, 28, 29, or 30 nucleotides in length.

“Nucleic acid” refers to deoxyribonucleotides or ribonucleotides and polymers thereof in single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

“Large double-stranded RNA” refers to any double-stranded RNA having a size greater than about 40 base pairs (bp) for example, larger than 100 bp or more particularly larger than 300 bp. The sequence of a large dsRNA may represent a segment of a mRNA or the entire mRNA. The maximum size of the large dsRNA is not limited herein. The double-stranded RNA may include modified bases where the modification may be to the phosphate sugar backbone or to the nucleoside. Such modifications may include a nitrogen or sulfur heteroatom or any other modification known in the art.

The double-stranded structure may be formed by self-complementary RNA strand such as occurs for a hairpin or a micro RNA or by annealing of two distinct complementary RNA strands.

“Overlapping” refers to when two RNA fragments have sequences which overlap by a plurality of nucleotides on one strand, for example, where the plurality of nucleotides (nt) numbers as few as 2-5 nucleotides or by 5-10 nucleotides or more.

“One or more dsRNAs” refers to dsRNAs that differ from each other on the basis of sequence.

“Target gene or mRNA” refers to any gene or mRNA of interest. Indeed any of the genes previously identified by genetics or by sequencing may represent a target. Target genes or mRNA may include developmental genes and regulatory genes as well as metabolic or structural genes or genes encoding enzymes. The target gene may be expressed in those cells in which a phenotype is being investigated or in an organism in a manner that directly or indirectly impacts a phenotypic characteristic. The target gene may be endogenous or exogenous. Such cells include any cell in the body of an adult or embryonic animal or plant including gamete or any isolated cell such as occurs in an immortal cell line or primary cell culture.

In this specification and the appended claims, the singular forms of “a”, “an” and “the” include plural reference unless the context clearly dictates otherwise.

The polypeptide PN73 represents a partial amino acid sequence corresponding at least in part to a partial sequence of a histone protein, for example of one or more of the following histones: histone H1, histone H2A, histone H2B, histone H3 or histone H4, or one or more polypeptide fragments or derivatives thereof comprising at least a partial sequence of a histone protein, typically at least 5-10 or 10-20 contiguous residues of a native histone protein. In exemplary embodiments, the histone polynucleotide delivery-enhancing polypeptide comprises a fragment of histone H2B, as exemplified by the polynucleotide delivery-enhancing polypeptide designated PN73 described herein below. In yet additional detailed embodiments, the polynucleotide delivery-enhancing polypeptide may be pegylated to improve stability and/or efficacy, particularly in the context of in vivo administration. The amino acid sequence of PN73 is shown below and it has a molecular weight of 4229.1 Daltons:

KGSKKAVTKAQKKDGKKRKRSRKESYSVYVYKVLKQ

Within additional embodiments of the invention, the polynucleotide delivery-enhancing polypeptide is selected or rationally designed to comprise an amphipathic amino acid sequence. For example, useful polynucleotide delivery-enhancing polypeptides may be selected which comprise a plurality of non-polar or hydrophobic amino acid residues that form a hydrophobic sequence domain or motif, linked to a plurality of charged amino acid residues that form a charged sequence domain or motif, yielding an amphipathic peptide.

In other embodiments, the polynucleotide delivery-enhancing polypeptide is selected to comprise a protein transduction domain or motif, and a fusogenic peptide domain or motif. A protein transduction domain is a peptide sequence that is able to insert into and preferably transit through the membrane of cells. A fusogenic peptide is a peptide that is able destabilize a lipid membrane, for example a plasma membrane or membrane surrounding an endosome, which may be enhanced at low pH. Exemplary fusogenic domains or motifs are found in a broad diversity of viral fusion proteins and in other proteins, for example fibroblast growth factor 4 (FGF4).

To rationally design polynucleotide delivery-enhancing polypeptides of the invention, a protein transduction domain is employed as a motif that will facilitate entry of the nucleic acid into a cell through the plasma membrane. In certain embodiments, the transported nucleic acid will be encapsulated in an endosome. The interior of endosomes has a low pH resulting in the fusogenic peptide motif destabilizing the membrane of the endosome. The destabilization and breakdown of the endosome membrane allows for the release of the siNA into the cytoplasm where the siNA can associate with a RISC complex and be directed to its target mRNA.

Examples of protein transduction domains for optional incorporation into polynucleotide delivery-enhancing polypeptides of the invention include:

| | |
|---|---|
| 1. | TAT protein transduction domain (PTD) (SEQ ID |
| | NO:1) KRRQRRR; |
| 2. | Penetratin PTD (SEQ ID NO:2) RQIKIWFQNRRMKWKK; |
| 3. | VP22 PTD (SEQ ID NO:3) DAATATRGRSAASRPTERPRAPAR |
| | SASRPRRPVD; |
| 4. | Kaposi FGF signal sequences (SEQ ID NO:4) AAVAL |
| | LPAVLLALLAP, and SEQ ID NO:5) AAVLLPVLLPVLLAAP; |
| 5. | Human β3 integrin signal sequence (SEQ ID NO: |
| | 6) VTVLALGALAGVGVG; |
| 6. | gp41 fusion sequence (SEQ ID NO:7) GALFLGWLGAAG |
| | STMGA; |
| 7. | Caiman crocodylus Ig(v) light chain (SEQ ID NO: |
| | 8) MGLGLHLLVLAAALQGA; |
| 8. | hCT-derived peptide (SEQ ID NO:9) LGTYTQDFNKFHT |
| | FPQTAIGVGAP; |
| 9. | Transportan (SEQ ID NO:10) GWTLNSAGYLLKINLKALAA |
| | LAKKIL; |
| 10. | Loligomer (SEQ ID NO:11) TPPKKKRKVEDPKKKK; |
| 11. | Arginine peptide (SEQ ID NO:12) RRRRRRR; |
| and | |
| 12. | Amphiphilic model peptide (SEQ ID NO:13) KLALKL |
| | ALKALKAALKLA. |

Examples of viral fusion peptides fusogenic domains for optional incorporation into polynucleotide delivery-enhancing polypeptides of the invention include:

| | |
|---|---|
| 1. | Influenza HA2 (SEQ ID NO:14) GLFGAIAGFIENGWEG; |
| 2. | Sendai F1 (SEQ ID NO:15) FFGAVIGTIALGVATA; |
| 3. | Respiratory Syncytial virus F1 (SEQ ID NO:16) |
| | FLGFLLGVGSAIASGV; |
| 4. | HIV gp41 (SEQ ID NO:17) GVFVLGFLGFLATAGS; |
| and | |
| 5. | Ebola GP2 (SEQ ID NO:18) GAAIGLAWIPYFGPAA. |

Within yet additional embodiments of the invention, polynucleotide delivery-enhancing polypeptides are provided that incorporate a DNA-binding domain or motif which facilitates polypeptide-siNA complex formation and/or enhances delivery of siNAs within the methods and compositions of the invention. Exemplary DNA binding domains in this context include various “zinc finger” domains as described for DNA-binding regulatory proteins and other proteins identified in Table 1, below (see, e.g., Simpson et al., J. Bio. Chem. 278:28011-28018, 2003).

TABLE 1
Exemplary Zinc Finger Motifs of Different DNA-Binding Proteins

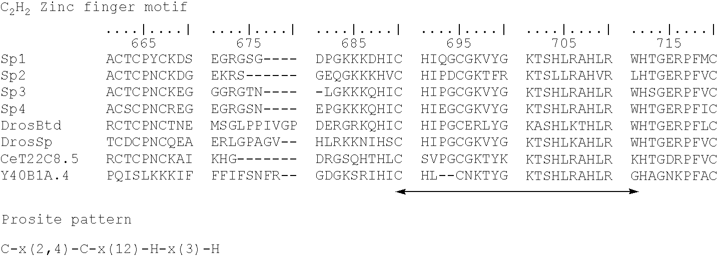

* The table demonstrates a conservative zinc fingerer motif for double strand DNA binding which is characterized by the C-x(2,4)-C-x(12)-H-x(3)-H motif pattern, which itself can be used to select and design additional polynucleotide delivery-enhancing polypeptides according to the invention.
** The sequences shown in Table 1, for Sp1, Sp2, Sp3, Sp4, DrosBtd, DrosSp, CeT22C8.5, and Y4pB1A.4, are herein assigned SEQ ID NO:s 19, 20, 21, 22, 23, 24, 25, and 26, respectively.

Alternative DNA binding domains useful for constructing polynucleotide delivery-enhancing polypeptides of the invention include, for example, portions of the HIV Tat protein sequence (see Examples below).

Within exemplary embodiments of the invention described herein below, polynucleotide delivery-enhancing polypeptides may be rationally designed and constructed by combining any of the foregoing structural elements, domains or motifs into a single polypeptide effective to mediate enhanced delivery of siNAs into target cells. For example, a protein transduction domain of the TAT polypeptide was fused to the N-terminal 20 amino acids of the influenza virus hemagglutinin protein, termed HA2, to yield one exemplary polynucleotide delivery-enhancing polypeptide herein. Various other polynucleotide delivery-enhancing polypeptide constructs are provided in the instant disclosure, evincing that the concepts of the invention are broadly applicable to create and use a diverse assemblage of effective polynucleotide delivery-enhancing polypeptides for enhancing siNA delivery.

Yet additional exemplary polynucleotide delivery-enhancing polypeptides within the invention may be selected from the following peptides: WWETWKPFQCRICMRNFSTRQARRNHRRRHR (SEQ ID NO: 27); GKINLKALAALAKKIL (SEQ ID NO: 28), RVIRVWFQNKRCKDKK (SEQ ID NO: 29), GRKKRRQRRRPPQGRKKRRQRRRPPQGRKKRRQRRRPPQ (SEQ ID NO: 30), GEQIAQLIAGYIDIILKKKKSK (SEQ ID NO: 31), Poly Lys-Trp, 4:1, MW 20,000-50,000; and Poly Orn-Trp, 4:1, MW 20,000-50,000. Additional polynucleotide delivery-enhancing polypeptides that are useful within the compositions and methods herein comprise all or part of the mellitin protein sequence.

Charged Molecules

Examples of organic cations for use within the invention include, but are not limited to: ammonium hydroxide, D-arginine, L-arginine, t-butylamine, calcium DL-malate, choline, dethanolamine, ethylenediamine, glycine, L-histidine, L-lysine, magnesium hydroxide, N-methyl-D-glucamine, L-ornithine hydrochloride, procaine hydrochloride, L-proline, pyridoxine, L-serine, sodium hydroxide, DL-triptophan, tromethamine, L-tyrosine, L-valine, carnitine, taurine, creatine malate, arginine alpha keto glutarate, ornithine alpha keto glutarate, spermine acetate, and spermidine chloride.

Other examples include aliphatic amines such as primary, secondary, tertiary and quaternary ammonium compounds from C4 through C22 aliphatics, preferably even numbered aliphatic hydrocarbons. Examples of primary aliphatic amines include N-propylamine; N-butylamine; N-pentylamine; N-hexylamine, N-heptylamine, N-octylamine, N-nonylamine; N-decylamine, N-dodecylamine; N-tetradecylamine; N-hexadecylamine, and N-octadecylamine.

Examples of secondary aliphatic amines include N,N-dipropylamine, N,N-dibutylamine; N,N-dipentylamine; N,N-dihexylamine, N,N-diheptylamine, N,N-dioctylamine, N,N-dinonylamine; N,N-didecylamine, N,N-didodecylamine; N,N,-ditetradecylamine; N,N-dihexadecylamine, and N,N-dioctadecylamine.

Examples of quaternary ammonium straight chain aliphatic amines include N-propyl-N,N,N-trimethylammonium chloride; N-butyl-N,N,N-trimethylammonium chloride, N-pentyl-N,N,N-trimethylammonium chloride; N-hexyl-N,N,N-trimethylammonium chloride; N-heptyl-N,N,N-trimethylammonium chloride; N-octyl-N,N,N-trimethylammonium chloride; N-nonyl-N,N,N-trimethylammonium chloride; N-decyl-N,N,N-trimethylammonium chloride; N-dodecyl-N,N,N-trimethylammonium chloride; N-tetradecyl-N,N,N-trimethylammonium chloride; N-hexadecyl-N,N,N-trimethylammonium chloride; and N-octadecyl-N,N,N-trimethylammonium chloride.

Examples of disubstituted symmetric quaternary aliphatic amines include N,N-dipropyl-N,N-dimethylammonium chloride; N,N-dibutyl-N,N-dimethylammonium chloride, N,N-dipentyl-N,N-dimethylammonium chloride; N,N-dihexyl-N,N-diimethylammonium chloride; N,N-diheptyl-N,N-diimethylammonium chloride; N,N-dioctyl-N,N-dimethylammonium chloride; N,N-dinonyl-N,N-dimethylammonium chloride; N,N-didecyl-N,N-dimethylammonium chloride; N,N-didodecyl-N,N-dimethylammonium chloride; N,N-ditetradecyl-N,N-dimethylammonium chloride; N,N-dihexadecyl-N,N-dimethylammonium chloride; and N,N-dioctadecyl-N,N-dimethylammonium chloride (DDAB).

Examples of unsaturated aliphatic quaternary ammonium cationic surfactant enes include N-1-propene-N,N,N-trimethylammonium chloride; N-1-butene-N,N,N-trimethylammonium chloride, N-1-pentene-N,N,N-trimethylammonium chloride; N-hexene-N,N,N-trimethylammonium chloride; N-heptene-N,N,N-trimethylammonium chloride; N-octene-N,N,N-trimethylammonium chloride; N-nonene-N,N,N-trimethylammonium chloride; N-decene-N,N,N-trimethylammonium chloride; N-dodecene-N,N,N-trimethylammonium chloride; N-tetradecene-N,N,N-trimethylammonium chloride; N-hexadecene-N,N,N-trimethylammonium chloride; and N-oleylamine-N,N,N-trimethylammonium chloride.

Examples of disubstituted symmetric quaternary unsaturated aliphatic amines include N,N-di-1-propene-N,N-dimethylammonium chloride; N,N-di-1-butene-N,N-dimethylammonium chloride, N,N-di-1-pentene-N,N-dimethylammonium chloride; N,N-dihexene-N,N-dimethylammonium chloride; N,N-diheptene-N,N-dimethylammonium chloride; N,N-dioctene-N,N-dimethylammonium chloride; N,N-dinonene-N,N-dimethylammonium chloride; N,N-decene-N,N-dimethylammonium chloride; N,N-didodecene-N,N-dimethylammonium chloride; N,N-tetradecene-N,N-dimethylammonium chloride; N,N-dihexadecene-N,N-dimethylammonium chloride; and N,N-dioleylamine-N,N-dimethylammonium chloride.

Other examples of organic cations include: DOTMA (N-[1-(2,3-dioleoyloxy) propyl]-N,N,N-trimethylammonium chloride, U.S. Pat. No. 4,897,355, DMRIE (D,L-1,2-0-dimyristyl-3-dimethylaminopropyl-b-hydroxyethylammoniumchloride, U.S. Pat. No. 5,264,618, DOTAP (1,2-bis (oleoyloxy)-3-3(trimethylammonia) propane) (Boehringer-Mannheim Catalog No. 1 202 375), DOGS (5-carboxysperminyglycine dioctadecylamide, U.S. Pat. No. 5,171,678 (TRANSFECTAM, Promega Corp., Madison, Wis.), DOSPA (2,3-dioleyloxy-N-[2(sperminecarboxyamido)ethyl}-N,N-dimethyl-1-propanaminium trifluoroacetate, U.S. Pat. No. 5,334,761, DDAB (Dimethyloctadecylammoniumbromide, U.S. Pat. No. 5,279,833, and TMTPS (N,N,N,N-Tetramethyltetrapalmylspermine, PCT Int. Pub. No. WO 95/17373. DOTMA, DOSPA, DDAB and TMTPS are sold by Life Technologies, Inc., Gaithersburg, Md., under the trade names LIPOFECTIN, LIPOFECTAMINE, LIPOFECTACE and CELLFECTIN,respectively. Other examples of organic cations are given in Ruysschaert et al., Biochem. Biophys. Res., 1994.

Examples of organic anions for use within the invention include, but are not limited to: acetic acid, adamantoic acid, alpha keto glutaric acid, D-aspartic acid, L-aspartic acid, benzenesulfonic acid, benzoic acid, 10-camphorsulfunic acid, citric acid, 1,2-ethanedisulfonic acid, fumaric acid, D-gluconic acid, D-glucuronic acid, glucaric acid, D-glutamic acid, L-glutamic acid, glutaric acid, glycolic acid, hippuric acid, hydrobromic acid, hydrochloric acid, 1-hydroxyl-2-napthoic acid, lactobioinic acid, maleic acid, L-malic acid, mandelic acid, methanesulfonic acid, mucic acid, 1,5napthalenedisulfonic acid tetrahydrate, 2-napthalenesulfonic acid, nitric acid, oleic acid, pamoic acid, p-toluenesulfonic acid hydrate, D-saccharic acid monopotassium salt, salicylic acid, stearic acid, succinic acid, sulfuric acid, tannic acid, D-tartaric acid, L-tartaric acid, and other relate sugar carboxylate anions.

### EXAMPLES

The invention will be further described in the following examples, which do not limit the scope of the invention described in the claims. A crucial factor in the administration of any therapeutic agent in an aqueous formulation is solubility. In light of this, quite often the concentration required to administer a single therapeutically effect dose in a reasonable volume to patients exceeds the inherent solubility characteristics of the therapeutic agent. The following examples describe methods and formulations employed to create stable siNA/polynucleotide delivery-enhancing polypeptide complexes in solution.

### Example 1

### Low Concentrations of LC20 siRNA/PN73

### Peptide Complex Precipitate Readily from Solution

The present example exemplifies the intrinsic instability of the LC20 siRNA/PN73 peptide complex at a concentration of 100 μM in a phosphate buffered saline (PBS) solution. The solution contains 250 μg/mL LC20 siRNA and 400 μg/mL PN73 peptide. Upon mixing LC20 siRNA and PN73 in PBS, this formulation immediately shows extensive turbidity and varied levels of precipitation with occlusive particulate contamination visible with the naked eye. In addition, characterization of the complex by static laser light scattering shows the presence of particular matter. As a result of the promiscuous aggregation of this complex, the LC20/PN73 complex is difficult to analyze by size exclusion chromatography. Lastly, a visible pellet is observed after centrifugation of the mixture, which is refractory to resuspension in water indicating the complex is highly insoluble. Analysis of the supernatant by UV spectrophotometry (UV260) shows a nearly 50-fold decrease in LC20 siRNA concentration in solution relative to the 250 μg/mL starting concentration. Taken together, these observations indicate that the LC20/PN73 complex undergoes extensive aggregation in solution and is not suitable for effective therapeutic administration.

In light of this inherent instability, the administration of this complex in aqueous form is problematic requiring that the LC20 siRNA and PN73 peptide be stored separately until the time of administration. Furthermore, delivery of this formulation to cells in culture or animal subjects requires that the separate components be mixed and immediately administered to avoid rendering the dose therapeutically ineffective due to aggregation and precipitation. Aggregation may be avoided with lower concentrations of LC20 siRNA and PN73 peptide. However, the resulting concentration would not be sufficient to accommodate a single dose in an appropriate volume for administration. As a consequence, an unreasonable number of administrations would be required to achieve a therapeutically effective dose potentially causing the patient or animal subject discomfort and non-compliance.

The following examples explain various compositions and methods that stabilize the LC20 siRNA/PN73 peptide complex in solution.

### Example 2

### The Addition of Various Organic Salt Competitors

### Creates LC20 siRNA/PN73 Peptide Complex Stability

In this example, the efficacy of various organic cationic and anionic competitors to create LC20 siRNA/PN73 peptide complex stability was tested. An intrinsic characteristic of the PN73 peptide is to aggregate and form large complexes. The addition of the LC20 siRNA reduces this aggregation; however, it does not prevent it nor reduce it significantly. Thus, an array of candidate organic cationic and anionic competitors were tested to determine if they could further reduce aggregation and promote LC20 siRNA/PN73 peptide complex stability in solution.

The ability of the organic salt competitor to promote complex stability was determined by the presence or absence of particle formation as measured by the naked eye. A visibly clear solution indicated that the salt competitor created LC20 siRNA/PN73 peptide complex stability. Further, all samples were analyzed by size exclusion chromatography coupled with an ultraviolet (UV) detector and a static laser light scattering detector (see Example 3). All experiments were performed in a final volume of 0.5 mL to 2.0 mL phosphate buffered saline at pH 7.2 with 17.5 μM LC20 siRNA and 95 μM PN73 (5:1 stiochiometry of PN73 peptide to LC20 siRNA). The working concentration of the LC20 siRNA/PN73 peptide complex was 100 μM.

This study examines whether the order of addition of the LC20 siRNA and the PN73 peptide to the organic salt competitor is a factor in maximizing LC20 siRNA/PN73 peptide complex stability. The following organic cations were used in this study: N-methyl-D-glucamine (NMDG), trimethylethanolamine (Choline), arginine, and spermine. They were chosen because they are well characterized and known to be safe for pharmaceutical salts. NMDG and arginine were tested with a glutamate anion while trimethylethanolamine was tested with a chloride anion. Spermine was tested with an acetate anion. Each salt was tested at a 100 mM, 10 mM and 1 mM concentration. This concentration range was chosen to promote stability for siRNA/PN73 and provide for an isotonic solution.

The PN73 peptide was mixed with 100 mM, 10 mM or 1 mM of the salt competitor followed by the addition of the LC20 siRNA. The contra experiment was performed whereby the LC20 siRNA was mixed first with the organic salt competitor followed then by the addition of the PN73 peptide. Both methods resulted in a clear solution indicating that the tested salt competitors can prevent LC20 siRNA/PN73 peptide complex aggregation and the order of addition of the organic salt competitor is not relevant to maximize complex stability in solution.

### Example 3

### Physical Characterization of the Organic

### Salt with the LC20 siRNA/PN73 Peptide Complex

In this example, size exclusion chromatography (SEC) coupled with an ultraviolet detector (UV 260nm) and static laser light scattering (LS) detector was used to characterize the physical properties of the LC20 siRNA/PN73 peptide complex in the presence or absence of the organic salt. In addition, the phosphate/nitrogen (P/N) charge ratio for LC20 siRNA/PN73 was calculated.

Size Exclusion Chromatography/UV Detection/LS Detection

PN73 in monomeric form is 4 kiloDaltons (kDA); however an intrinsic property of this peptide is to aggregate and form large complexes in solution. An initial study was performed to analyze the physical properties of PN73, without LC20 siRNA, in the presence and absence of 100 mM NMDG-glutamate salt or 9% sorbitol (no salt environment). In the presence of 9% sorbitol, a UV trace with two overlapping peaks was observed at approximately 9 minutes. The LS signal showed that the molecular weight of the species that eluted from the size exclusion column was approximately 3 megaDaltons indicating that a significant amount of aggregation occurred after PN73 passed through the size exclusion column. In contrast, in the presence of 100 mM NMDG-glutamate salt, two distinct adjacent UV traces were observed indicating two distinct species of PN73 were present. The LS signal indicated that one species was approximately 3 megaDaltons, representing a large PN73 aggregate, and the other approximately 40 kDa. The 40 kDa molecular weight species indicates that the presence of 100 mM NMDG-glutamate salt reduces PN73 aggregation significantly. Next, the ability of NMDG-glutamate and other organic salts to reduced PN73 aggregation in the presence of LC20 siRNA was characterized by SEC-UV/LS.

LC20 siRNA/PN73 aggregation was characterized in the presence or absence of NMDG-glutamate by SEC-UV/LS. In the absence of NMDG-glutamate, a two overlapping UV traces were observed at 9 minutes which represented dissociated LC20 siRNA and PN73 molecules. In contrast, in the presence of 100 mM, 10 mM or 1 mM NMDG-glutamate, an additional UV trace was observed at approximately 5 minutes, indicating a stable LC20 siRNA/PN73 complex was present. The LS trace showed that a larger molecular weight species was created with LC20 siRNA and PN73 in the presence of NMDG-glutamate than in the absence of NMDG-glutamate. These data indicate that NMDG-glutamate is an effective stabilizer of the LC20 siRNA/PN73 complex in solution at concentrations of 100 mM, 10 mM, and 1 mM.

A similar SEC-UV/LS profile was observed with 100 mM, 10 mM, and 1 mM arginine glutamate indicating that, like NMDG-glutamate, arginine glutamate is an effective stabilizer at these salt concentrations. However, the LS trace for 150 mM arginine glutamate showed a significant presence of intermediary aggregating molecules between the 9 minute and 5 minute UV traces. Thus, arginine glutamate is not an effective stabilizer at a 150 mM concentration.

Spermine acetate at 10 mM and 1 mM showed a similar SEC-UV/LS profile indicating it too is an effective LC20 siRNA/PN73 stabilizer at 10 mM and 1 mM. In contrast, LC20 siRNA and PN73 in the presence of 100 mM spermine acetate showed an additional UV trace at approximately 7 minutes and a significantly reduced UV trace at 5 minutes (i.e., the peak corresponding to a stable LC20 siRNA/PN73 complex). This data indicates that 100 mM spermine acetate dissociates the LC20 siRNA/PN73 peptide complex. Thus, spermine acetate is an effective stabilizer of the LC20 siRNA/PN73 complex at a concentration of more than 1 mM but less than 100 mM.

Choline chloride showed UV traces similar to the other organic salts tested; however, the LS trace for choline chloride at 100 mM, 10 mM and 1 mM showed a significant presence of intermediary aggregating molecules between the 9 minute and 5 minute UV traces. Therefore, choline chloride can stabilize the LC20 siRNA/PN73 peptide complex, but it also allows for the formation of unwanted aggregates in solution. One interpretation of this is that choline chloride prevents LC20 siRNA/PN73 peptide complex aggregation in a time dependent manner. Nonetheless, it may not be suitable as a stabilizer at the concentrations tested.

Charge Ratio Calculations for LC20/PN73

The phosphate (P) to nitrogen (N) charge ratio (P/N) was calculated for the LC20/PN73 complex. The molar concentration of phosphate anions in LC20 siRNA was calculated to be 720 μM or 0.72 mM (P) and the molar concentration of the protonated nitrogen cations in PN73 was calculated to be 1.23 mM. At a 1:1 stiochiometry, all LC20 siRNA/PN73 peptide conjugates have a P/N ratio of 3 indicating that the complex forms large aggregates over time making it ineffective as delivery agent. However, as presented in the above Examples, the addition of cationic and anionic salts with LC20 siRNA/PN73 prevents aggregations and promotes complex stability in solution.

1. A method for producing a complex between a double stranded (ds) nucleic acid and a peptide, comprising:
a. solubilizing the nucleic acid in an aqueous solution;
b. solubilizing the peptide in an aqueous solution; and
c. mixing the solubilized ds nucleic acid and the solubilized peptide in the presence of an organic salt, wherein steps a, b and c may be performed in any order, or simultaneously.

2. The method of claim 1, wherein the ds nucleic acid is a dsRNA.

3. The method of claim 2, wherein the dsRNA is a siRNA.

4. The method of claim 3, wherein the siRNA has 29-50 base pairs.

5. The method of claim 4, wherein the siRNA is comprised of a sequence that is complementary to a region of a TNF-alpha gene.

6. The method of claim 1, wherein the ds nucleic acid is a dsDNA.

7. The method of claim 1, wherein the peptide is a polynucleotide delivery-enhancing polypeptide.

8. The method of claim 7, wherein the polynucleotide delivery-enhancing polypeptide comprises a histone protein, or a polypeptide or peptide fragment, derivative, analog, or conjugate thereof.

9. The method of claim 7, wherein the polynucleotide delivery-enhancing polypeptide comprises an amphipathic amino acid sequence.

10. The method of claim 7, wherein the polynucleotide delivery-enhancing polypeptide comprises a protein transduction domain or motif.

11. The method of claim 7, wherein the polynucleotide delivery-enhancing polypeptide comprises a fusogenic peptide domain or motif.

12. The method of claim 7, wherein the polynucleotide delivery-enhancing polypeptide comprises a nucleic acid-binding domain or motif.

13. The method of claim 12, wherein the polypeptide binds ds nucleic acid with a Kd less than about 100 nM.

14. The method of claim 12, wherein the polypeptide binds ds nucleic acid with a Kd less than about 10 nM.

15. The method of claim 7, wherein the polynucleotide delivery-enhancing polypeptide selected from the group consisting of:
(SEQ ID NO:32)
GRKKRRQRRRPPQC
(SEQ ID NO:33)
Maleimide-AAVALLPAVLLALLAPRKKRRQRRRPPQ-amide
(SEQ ID NO:34)
AAVALLPAVLLALLAPRKKRRQRRRPPQC
(SEQ ID NO:35)
Maleimide-AAVALLPAVLLALLAPRKKRRQRRRPPQ-amide
(SEQ ID NO:36)
NH2-RKKRRQRRRPPQCAAVALLPAVLLALLAP-amide
(SEQ ID NO:37)
BrAc-GRKKRRQRRRPQ-amide
(SEQ ID NO:38)
BrAc-RRRQRRKRGGDIMGEWGNEIFGAIAGFLGamide
(SEQ ID NO:39)
NH2-RRRQRRKRGGDIMGEWGNEIFGAIAGFLG-amide
(SEQ ID NO:40)
C(YGRKKRRQRRRG)2
(SEQ ID NO:41)
Maleimide-GRKKRRQRRRPPQ-amide
(SEQ ID NO:42)
NH2-KLWKAWPKLWKKLWKP-amide
(SEQ ID NO:43)
AAVALLPAVLLALLAPRRRRRR-amide
(SEQ ID NO:44)
RLWRALPRVLRRLLRP-amide
(SEQ ID NO:45)
NH2-AAVALLPAVLLALLAPSGASGLDKRDYV-amide
(SEQ ID NO:46)
Maleimide-AAVALLPAVLLALLAPSGASGLDKRDYV-amide
(SEQ ID NO:47)
NH2-SGASGLDKRDYVAAVAALLPAVLLALLAP-amide
(SEQ ID NO:48)
NH2-LLETLLKPFQCRICMRNFSTRQARRNHRRRHRR-amide
(SEQ ID NO:49)
NH2-AAVACRICMRNFSTRQARRNHRRRHRR-amide
(SEQ ID NO:50)
Maleimide-RQIKIWFQNRRMKWKK-amide
(SEQ ID NO:51)
RQIKIWFQNRRMKWKK amide
(SEQ ID NO:52)
NH2-RQIKIWFQNRRMKWKKDIMGEWGNEIFGAIAGFLG-amide
(SEQ ID NO:53)
Maleimide-SGRGKQGGKARAKAKTRSSRAGLQFPVGRVHRLLRKG-
amide
(SEQ ID NO:54)
SGRGKQGGKARAKAKTRSSRAGLQFPVGRVHRLLRKGC-amide
(SEQ ID NO:55)
KGSKKAVTKAQKKDGKKRKRSRK-amide
(SEQ ID NO:56)
NH2-KKDGKKRKRSRKESYSVYVYKVLKQ-amide
(SEQ ID NO:57)
KGSKKAVTKAQKKDGKKRKRSRKESYSVYVYKVLKQ
(SEQ ID NO:58)
BrAc-GWTLNSAGYLLGKINLKALAALAKKILamide
(SEQ ID NO:59)
KLALKLALKALKAALKLAamide
(SEQ ID NO:60)
BrAc-KLALKLALKALKAALKLAamide
(SEQ ID NO:61)
Ac-KETWWETWWTEWSQPKKKRKV-amide
(SEQ ID NO:62)
NH2-KETWWETWWTEWSQPGRKKRRQRRRPPQ-amide
(SEQ ID NO:63)
BrAc-RRRRRRR
(SEQ ID NO:64)
QqQqQqQqQq
(SEQ ID NO:65)
NH2-RRRQRRKRGGqQqQqQqQqQ-amide
(SEQ ID NO:66)
RVIRWFQNKRCKDKK-amide
(SEQ ID NO:67)
Ac-LGLLLRHLRHHSNLLANI-amide
(SEQ ID NO:68)
GQMSEIEAKVRTVKLARS-amide
(SEQ ID NO:69)
NH2-KLWSAWPSLWSSLWKP-amide
(SEQ ID NO:70)
NH2-KKKKKKKKK-amide
(SEQ ID NO:71)
NH2-AARLHRFKNKGKDSTEMRRRR-amide
(SEQ ID NO:72)
Maleimide-GLGSLLKKAGKKLKQPKSKRKV-amide
(SEQ ID NO:73)
Maleimide-Dmt-r-FK-amide
(SEQ ID NO:74)
Maleimide-Dmt-r-FKQqQqQqQqQq-amide
(SEQ ID NO:75)
Maleimide-WRFK-amide
(SEQ ID NO:76)
Maleimide-WRFKQqQqQqQqQq-amide
(SEQ ID NO:77)
Maleimido-YRFK-amide
(SEQ ID NO:78)
Maleimide-YRFKYRFKYRFK-amide
(SEQ ID NO:79)
Maleimide-WRFKKSKRKV-amide
(SEQ ID NO:80)
Maleimide-WRFKAAVALLPAVLLALLAP-amide
(SEQ ID NO:81)
NH2-DiMeYrFKamide
(SEQ ID NO:82)
NH2-YrFKamide
(SEQ ID NO:83)
NH2-DiMeYRFKamide
(SEQ ID NO:84)
NH2-WrFKamide
(SEQ ID NO:85)
NH2-DiMeYrWKamide
(SEQ ID NO:86)
NH2-KFrDiMeY-amide
(SEQ ID NO:87)
Maleimide-WRFKWRFK-amide
and
(SEQ ID NO:88)
Maleimide-WRFKWRFKWRFK-amide

16. The method of claim 7, wherein the polynucleotide delivery-enhancing polypeptide comprises one or more peptides selected from the group consisting of: histone H1, histone H2B, histone H3, and histone H4, or a histone fragment thereof;, GKINLKALAALAKKIL (SEQ ID NO: 89), RVIRVWFQNKRCKDKK (SEQ ID NO: 90), GRKKRRQRRRPPQGRKKRRQRRRPPQGRKKRRQRRRPPQ (SEQ ID NO: 91), GEQIAQLIAGYIDIILKKKKSK (SEQ ID NO: 92), WWETWKPFQCRICMRNFSTRQARRNHRRRHR (SEQ ID NO: 93), Poly Lys-Trp (4:1, MW 20,000-50,000), Poly Orn-Trp (4:1, MW 20,000-50,000), and mellitin.

17. The method of claim 15, wherein the polynucleotide delivery-enhancing polypeptide is KGSKKAVTKAQKKDGKKRKRSRKESYSVYVYKVLKQ (PN73) (SEQ ID NO: 94).

18. The method of claim 1, wherein the organic salt is selected from the group consisting of sugar amine and acid salts, peptide amine and acid salts, biocompatible salts, hydrophilic salts, and salts naturally occurring in man.

19. The method of claim 1, wherein the organic salt contains an organic cation.

20. The method of claim 19, wherein the organic cation is selected from the group consisting of N-propylamine; N-butylamine; N-pentylamine; N-hexylamine, N-heptylamine, N-octylamine, N-nonylamine; N-decylamine, N-dodecylamine; N-tetradecylamine; N-hexadecylamine, N-octadecylamine, N,N-dipropylamine, N,N-dibutylamine; N,N-dipentylamine; N,N-dihexylamine, N,N-diheptylamine, N,N-dioctylamine, N,N-dinonylamine; N,N-didecylamine, N,N-didodecylamine; N,N,-ditetradecylamine; N,N-dihexadecylamine, N,N-dioctadecylamine, N-propyl-N,N,N-trimethylammonium chloride; N-butyl-N,N,N-trimethylammonium chloride, N-pentyl-N,N,N-trimethylammonium chloride; N-hexyl-N,N,N-trimethylammonium chloride; N-heptyl-N,N,N-trimethylammonium chloride; N-octyl-N,N,N-trimethylammonium chloride; N-nonyl-N,N,N-trimethylammonium chloride; N-decyl-N,N,N-trimethylammonium chloride; N-dodecyl-N,N,N-trimethylammonium chloride; N-tetradecyl-N,N,N-trimethylammonium chloride; N-hexadecyl-N,N,N-trimethylammonium chloride; N-octadecyl-N,N,N-trimethylammonium chloride; N,N-dipropyl-N,N-dimethylammonium chloride; N,N-dibutyl-N,N-dimethylammonium chloride, N,N-dipentyl-N,N-dimethylammonium chloride; N,N-dihexyl-N,N-diimethylammonium chloride; N,N-diheptyl-N,N-diimethylammonium chloride; N,N-dioctyl-N,N-dimethylammonium chloride; N,N-dinonyl-N,N-dimethylammonium chloride; N,N-didecyl-N,N-dimethylammonium chloride; N,N-didodecyl-N,N-dimethylammonium chloride; N,N-ditetradecyl-N,N-dimethylammonium chloride; N,N-dihexadecyl-N,N-dimethylammonium chloride; N,N-dioctadecyl-N,N-dimethylammonium chloride (DDAB); N-1-propene-N,N,N-trimethylammonium chloride; N-1-butene-N,N,N-trimethylammonium chloride, N-1-pentene-N,N,N-trimethylammonium chloride; N-hexene-N,N,N-trimethylammonium chloride; N-heptene-N,N,N-trimethylammonium chloride; N-octene-N,N,N-trimethylammonium chloride; N-nonene-N,N,N-trimethylammonium chloride; N-decene-N,N,N-trimethylammonium chloride; N-dodecene-N,N,N-trimethylammonium chloride; N-tetradecene-N,N,N-trimethylammonium chloride; N-hexadecene-N,N,N-trimethylammonium chloride; N-oleylamine-N,N,N-trimethylammonium chloride; N,N-di-1-propene-N,N-dimethylammonium chloride; N,N-di-1-butene-N,N-dimethylammonium chloride, N,N-di-1-pentene-N,N-dimethylammonium chloride; N,N-dihexene-N,N-dimethylammonium chloride; N,N-diheptene-N,N-dimethylammonium chloride; N,N-dioctene-N,N-dimethylammonium chloride; N,N-dinonene-N,N-dimethylammonium chloride; N,N-decene-N,N-dimethylammonium chloride; N,N-didodecene-N,N-dimethylammonium chloride; N,N-tetradecene-N,N-dimethylammonium chloride; N,N-dihexadecene-N,N-dimethylammonium chloride; and N,N-dioleylamine-N,N-dimethylammonium chloride. N-[1-(2,3-dioleoyloxy)propyl]-N,N,N-trimethylammonium chloride, D,L-1,2-0-dimyristyl-3-dimethylaminopropyl-b-hydroxyethylammoniumchloride, 1,2-bis(oleoyloxy)-3-3(trimethylammonia)propane,5-carboxysperminyglycine dioctadecylamide, 2,3-dioleyloxy-N-[2(sperminecarboxyamido)ethyl}-N,N-dimethyl-1-propanaminium trifluoroacetate, Dimethyloctadecylammoniumbromide, and N,N,N,N-Tetramethyltetrapalmylspermine.

21. The method of claim 1, wherein the organic salt is selected from the group consisting of: D-arginine salt, L-arginine salt, t-butylamine salt, calcium DL-maleate, choline salt, ethanolamine salt, ethylenediamine salt, glycine salt, L-histidine salt, L-lysine salt, N-methyl-D-glucamine salt, L-ornithine hydrochloride, procaine hydrochloride, L-proline salt, pyridoxine salt, L-serine salt, DL-triptophan salt, tromethamine salt, L-tyrosine salt, L-valine salt, carnitine salt, taurine salt, creatine malate, arginine alpha keto glutarate, ornithine alpha keto glutarate, spermine acetate, and spermidine chloride.

22. The method of claim 1, wherein the organic salt is an organic anion.

23. The method of claim 1, wherein the organic salt is the salt of an acid selected from the group consisting of: acetic acid, adamantoic acid, alpha keto glutaric acid, D-aspartic acid, L-aspartic acid, benzenesulfonic acid, benzoic acid, 10-camphorsulfunic acid, citric acid, 1,2-ethanedisulfonic acid, fumaric acid, galacteric acid, D-gluconic acid, D-glucornic acid, glucaric acid, D-glutamic acid, L-glutamic acid, glutaric acid, glycolic acid, hippuric acid, hydrobromic acid, hydrochloric acid, 1-hydroxyl-2-napthoic acid, lactobiotinic acid, maleic acid, L-malic acid, mandelic acid, ethanesulfonic acid, mucic acid, 1,5napthalenedisulfonic acid tetrahydrate, 2-napthalenesulfonic acid, nitric acid, oleic acid, pamoic acid, p-toluenesulfonic acid hydrate, D-saccharic acid monopotassium salt, salicylic acid, steric acid, succinic acid, sulfuric acid, tannic acid, D-tartaric acid, L-tartaric acid, and other related sugar carboxylate anions.

24. A composition which is a complex between a double stranded (ds) nucleic acid and a peptide, comprising: the ds nucleic acid, the peptide and an organic salt, wherein the peptide binds the ds nucleic acid with a Kd less than about 100 nM, and wherein the complex is soluble in aqueous solution.

25. The composition of claim 24, wherein the ds nucleic acid is a dsRNA.

26. The composition of claim 25, wherein the dsRNA is a siRNA.

27. The composition of claim 26, wherein the siRNA has 29-50 base pairs.

28. The composition of claim 27, wherein the siRNA is comprised of a sequence that is complementary to a region of a TNF-alpha gene.

29. The composition of claim 24, wherein the ds nucleic acid is a dsDNA.

30. The composition of claim 24, wherein the peptide is a polynucleotide delivery-enhancing polypeptide.

31. The composition of claim 30, wherein the polynucleotide delivery-enhancing polypeptide comprises a histone protein, or a polypeptide or peptide fragment, derivative, analog, or conjugate thereof.

32. The composition of claim 30, wherein the polynucleotide delivery-enhancing polypeptide comprises an amphipathic amino acid sequence.

33. The composition of claim 30, wherein the polynucleotide delivery-enhancing polypeptide comprises a protein transduction domain or motif.

34. The composition of claim 30, wherein the polynucleotide delivery-enhancing polypeptide comprises a fusogenic peptide domain or motif.

35. The composition of claim 30, wherein the polynucleotide delivery-enhancing polypeptide comprises a nucleic acid-binding domain or motif.

36. The composition of claim 35, wherein the peptide binds ds nucleic acid with a Kd less than about 100 nM.

37. The composition of claim 36, wherein the peptide binds ds nucleic acid with a Kd less than about 10 nM.

38. The composition of claim 30, wherein the polynucleotide delivery-enhancing polypeptide selected from the group consisting of:
(SEQ ID NO:32)
GRKKRRQRRRPPQC
(SEQ ID NO:33)
Maleimide-AAVALLPAVLLALLAPRKKRRQRRRPPQ-amide
(SEQ ID NO:34)
AAVALLPAVLLALLAPRKKRRQRRRPPQC
(SEQ ID NO:35)
Maleimide-AAVALLPAVLLALLAPRKKRRQRRRPPQ-amide
(SEQ ID NO:36)
NH2-RKKRRQRRRPPQCAAVALLPAVLLALLAP-amide
(SEQ ID NO:37)
BrAc-GRKKRRQRRRPQ-amide
(SEQ ID NO:38)
BrAc-RRRQRRKRGGDIMGEWGNEIFGAIAGFLGamide
(SEQ ID NO:39)
NH2-RRRQRRKRGGDIMGEWGNEIFGAIAGFLG-amide
(SEQ ID NO:40)
C(YGRKKRRQRRRG)2
(SEQ ID NO:41)
Maleimide-GRKKRRQRRRPPQ-amide
(SEQ ID NO:42)
NH2-KLWKAWPKLWKKLWKP-amide
(SEQ ID NO:43)
AAVALLPAVLLALLAPRRRRRR-amide
(SEQ ID NO:44)
RLWRALPRVLRRLLRP-amide
(SEQ ID NO:45)
NH2-AAVALLPAVLLALLAPSGASGLDKRDYV-amide
(SEQ ID NO:46)
Maleimide-AAVALLPAVLLALLAPSGASGLDKRDYV-amide
(SEQ ID NO:47)
NH2-SGASGLDKRDYVAAVAALLPAVLLALLAP-amide
(SEQ ID NO:48)
NH2-LLETLLKPFQCRICMRNFSTRQARRNHRRRHRR-amide
(SEQ ID NO:49)
NH2-AAVACRICMRNFSTRQARRNHRRRHRR-amide
(SEQ ID NO:50)
Maleimide-RQIKIWFQNRRMKWKK-amide
(SEQ ID NO:51)
RQIKIWFQNRRMKWKK amide
(SEQ ID NO:52)
NH2-RQIKIWFQNRRMKWKKDIMGEWGNEIFGAIAGFLG-amide
(SEQ ID NO:53)
Maleimide-SGRGKQGGKARAKAKTRSSRAGLQFPVGRVHRLLRKG-
amide
(SEQ ID NO:54)
SGRGKQGGKARAKAKTRSSRAGLQFPVGRVHRLLRKGC-amide
(SEQ ID NO:55)
KGSKKAVTKAQKKDGKKRKRSRK-amide
(SEQ ID NO:56)
NH2-KKDGKKRKRSRKESYSVYVYKVLKQ-amide
(SEQ ID NO:57)
KGSKKAVTKAQKKDGKKRKRSRKESYSVYVYKVLKQ
(SEQ ID NO:58)
BrAc-GWTLNSAGYLLGKINLKALAALAKKILamide
(SEQ ID NO:59)
KLALKLALKALKAALKLAamide
(SEQ ID NO:60)
BrAc-KLALKLALKALKAALKLAamide
(SEQ ID NO:61)
Ac-KETWWETWWTEWSQPKKKRKV-amide
(SEQ ID NO:62)
NH2-KETWWETWWTEWSQPGRKKRRQRRRPPQ-amide
(SEQ ID NO:63)
BrAc-RRRRRRR
(SEQ ID NO:64)
QqQqQqQqQq
(SEQ ID NO:65)
NH2-RRRQRRKRGGqQqQqQqQqQ-amide
(SEQ ID NO:66)
RVIRWFQNKRCKDKK-amide
(SEQ ID NO:67)
Ac-LGLLLRHLRHHSNLLANI-amide
(SEQ ID NO:68)
GQMSEIEAKVRTVKLARS-amide
(SEQ ID NO:69)
NH2-KLWSAWPSLWSSLWKP-amide
(SEQ ID NO:70)
NH2-KKKKKKKKK-amide
(SEQ ID NO:71)
NH2-AARLHRFKNKGKDSTEMRRRR-amide
(SEQ ID NO:72)
Maleimide-GLGSLLKKAGKKLKQPKSKRKV-amide
(SEQ ID NO:73)
Maleimide-Dmt-r-FK-amide
(SEQ ID NO:74)
Maleimide-Dmt-r-FKQqQqQqQqQq-amide
(SEQ ID NO:75)
Maleimide-WRFK-amide
(SEQ ID NO:76)
Maleimide-WRFKQqQqQqQqQq-amide
(SEQ ID NO:77)
Maleimido-YRFK-amide
(SEQ ID NO:78)
Maleimide-YRFKYRFKYRFK-amide
(SEQ ID NO:79)
Maleimide-WRFKKSKRKV-amide
(SEQ ID NO:80)
Maleimide-WRFKAAVALLPAVLLALLAP-amide
(SEQ ID NO:81)
NH2-DiMeYrFKamide
(SEQ ID NO:82)
NH2-YrFKamide
(SEQ ID NO:83)
NH2-DiMeYRFKamide
(SEQ ID NO:84)
NH2-WrFKamide
(SEQ ID NO:85)
NH2-DiMeYrWKamide
(SEQ ID NO:86)
NH2-KFrDiMeY-amide
(SEQ ID NO:87)
Maleimide-WRFKWRFK-amide
and
(SEQ ID NO:88)
Maleimide-WRFKWRFKWRFK-amide

39. The composition of claim 38, wherein the polynucleotide delivery-enhancing polypeptide comprises one or more peptides selected from the group consisting of: histone H1, histone H2B, histone H3, and histone H4, or a histone fragment thereof;, GKINLKALAALAKKIL (SEQ ID NO: 89), RVIRVWFQNKRCKDKK (SEQ ID NO: 90), GRKKRRQRRRPPQGRKKRRQRRRPPQGRKKRRQRRRPPQ (SEQ ID NO: 91), GEQIAQLIAGYIDIILKKKKSK (SEQ ID NO: 92), WWETWKPFQCRICMRNFSTRQARRNHRRRHR (SEQ ID NO: 93), Poly Lys-Trp (4:1, MW 20,000-50,000), Poly Orn-Trp (4:1, MW 20,000-50,000), and mellitin.

40. The composition of claim 39, wherein the polypeptide is KGSKKAVTKAQKKDGKKRKRSRKESYSVYVYKVLKQ (PN73) (SEQ ID NO: 94).

41. The composition of claim 24, wherein the organic salt is selected from the group consisting of sugar amines and acids, peptide amines and acids, biocompatible salts, hydrophilic salts, and salts naturally occurring in man.

42. The composition of claim 41, wherein the organic salt contains an organic cation.

43. The composition of claim 42, wherein the organic cation is selected from the group consisting of N-propylamine; N-butylamine; N-pentylamine; N-hexylamine, N-heptylamine, N-octylamine, N-nonylamine; N-decylamine, N-dodecylamine; N-tetradecylamine; N-hexadecylamine, N-octadecylamine, N,N-dipropylamine, N,N-dibutylamine; N,N-dipentylamine; N,N-dihexylamine, N,N-diheptylamine, N,N-dioctylamine, N,N-dinonylamine; N,N-didecylamine, N,N-didodecylamine; N,N,-ditetradecylamine; N,N-dihexadecylamine, N,N-dioctadecylamine, N-propyl-N,N,N-trimethylammonium chloride; N-butyl-N,N,N-trimethylammonium chloride, N-pentyl-N,N,N-trimethylammonium chloride; N-hexyl-N,N,N-trimethylammonium chloride; N-heptyl-N,N,N-trimethylammonium chloride; N-octyl-N,N,N-trimethylammonium chloride; N-nonyl-N,N,N-trimethylammonium chloride; N-decyl-N,N,N-trimethylammonium chloride; N-dodecyl-N,N,N-trimethylammonium chloride; N-tetradecyl-N,N,N-trimethylammonium chloride; N-hexadecyl-N,N,N-trimethylammonium chloride; N-octadecyl-N,N,N-trimethylammonium chloride; N,N-dipropyl-N,N-dimethylammonium chloride; N,N-dibutyl-N,N-dimethylammonium chloride, N,N-dipentyl-N,N-dimethylammonium chloride; N,N-dihexyl-N,N-diimethylammonium chloride; N,N-diheptyl-N,N-diimethylammonium chloride; N,N-dioctyl-N,N-dimethylammonium chloride; N,N-dinonyl-N,N-dimethylammonium chloride; N,N-didecyl-N,N-dimethylammonium chloride; N,N-didodecyl-N,N-dimethylammonium chloride; N,N-ditetradecyl-N,N-dimethylammonium chloride; N,N-dihexadecyl-N,N-dimethylammonium chloride; N,N-dioctadecyl-N,N-dimethylammonium chloride (DDAB); N-1-propene-N,N,N-trimethylammonium chloride; N-1-butene-N,N,N-trimethylammonium chloride, N-1-pentene-N,N,N-trimethylammonium chloride; N-hexene-N,N,N-trimethylammonium chloride; N-heptene-N,N,N-trimethylammonium chloride; N-octene-N,N,N-trimethylammonium chloride; N-nonene-N,N,N-trimethylammonium chloride; N-decene-N,N,N-trimethylammonium chloride; N-dodecene-N,N,N-trimethylammonium chloride; N-tetradecene-N,N,N-trimethylammonium chloride; N-hexadecene-N,N,N-trimethylammonium chloride; N-oleylamine-N,N,N-trimethylammonium chloride; N,N-di-1-propene-N,N-dimethylammonium chloride; N,N-di-1-butene-N,N-dimethylammonium chloride, N,N-di-1-pentene-N,N-dimethylammonium chloride; N,N-dihexene-N,N-dimethylammonium chloride; N,N-diheptene-N,N-dimethylammonium chloride; N,N-dioctene-N,N-dimethylammonium chloride; N,N-dinonene-N,N-dimethylammonium chloride; N,N-decene-N,N-dimethylammonium chloride; N,N-didodecene-N,N-dimethylammonium chloride; N,N-tetradecene-N,N-dimethylammonium chloride; N,N-dihexadecene-N,N-dimethylammonium chloride; and N,N-dioleylamine-N,N-dimethylammonium chloride. N-[1-(2,3-dioleoyloxy)propyl]-N,N,N-trimethylammonium chloride, D,L-1,2-0-dimyristyl-3-dimethylaminopropyl-b-hydroxyethylammoniumchloride, 1,2-bis(oleoyloxy)-3-3(trimethylammonia)propane,5-carboxysperminyglycine dioctadecylamide, 2,3-dioleyloxy-N-[2(sperminecarboxyamido)ethyl}-N,N-dimethyl-1-propanaminium trifluoroacetate, Dimethyloctadecylammoniumbromide, and N,N,N,N-Tetramethyltetrapalmylspermine.

44. The composition of claim 41, wherein the organic salt is selected from the group consisting of: ammonium hydroxide, D-arginine, L-arginine, t-butylamine, calcium DL-malate, choline, ethanolamine, ethylenediamine, glycine, L-histidine, L-lysine, magnesium hydroxide, N-methyl-D-glucamine, L-ornithine hydrochloride, procaine hydrochloride, L-proline, pyridoxine, L-serine, sodium hydroxide, DL-triptophan, tromethamine, L-tyrosine, L-valine, carnitine, taurine, creatine malate, arginine alpha keto glutarate, ornithine alpha keto glutarate, spermine acetate, and spermidine chloride.

45. The composition of claim 41, wherein the organic salt contains an organic anion.

46. The composition of claim 45, wherein the organic salt is the salt of an acid selected from the group consisting of: acetic acid, adamantoic acid, alpha keto glutaric acid, D-aspartic acid, L-aspartic acid, benzenesulfonic acid, benzoic acid, 10-camphorsulfunic acid, citric acid, 1,2-ethanedisulfonic acid, fumaric acid, galacteric acid, D-gluconic acid, D-glucornic acid, glucaric acid, D-glutamic acid, L-glutamic acid, glutaric acid, glycolic acid, hippuric acid, hydrobromic acid, hydrochloric acid, 1-hydroxyl-2napthoic acid, lactobiotinic acid, maleic acid, L-malic acid, mandelic acid, ethanesulfonic acid, mucic acid, 1,5 napthalenedisulfonic acid tetrahydrate, 2-napthalenesulfonic acid, nitric acid, oleic acid, pamoic acid, p-toluenesulfonic acid hydrate, D-saccharic acid monopotassium salt, salicylic acid, steric acid, succinic acid, sulfuric acid, tannic acid, D-tartaric acid, L-tartaric acid, and other related sugar carboxylate anions.
